SEQUENCE LISTING (14 sequences)

SEQ ID NO: 1 - RNA, 19 nt, Artificial Sequence
  Description of Artificial Sequence Synthetic oligonucleotide
cuguuauguagaacgcuga

SEQ ID NO: 2 - RNA, 19 nt, Artificial Sequence
  Description of Artificial Sequence Synthetic oligonucleotide
gcugcagcuuuguaugauu

SEQ ID NO: 3 - DNA, 20 bp, Artificial Sequence
  Description of Artificial Sequence Synthetic oligonucleotide
ggttcccaaggacctatatg

SEQ ID NO: 4 - DNA, 20 bp, Artificial Sequence
  Description of Artificial Sequence Synthetic oligonucleotide
acagagggcccggctgttga

SEQ ID NO: 5 - DNA, 20 bp, Artificial Sequence
  Description of Artificial Sequence Synthetic oligonucleotide
ccaaagctaaaaacttgatg

SEQ ID NO: 6 - DNA, 20 bp, Artificial Sequence
  Description of Artificial Sequence Synthetic oligonucleotide
caacagtgggcattgatttc

SEQ ID NO: 7 - DNA, 20 bp, Artificial Sequence
  Description of Artificial Sequence Synthetic oligonucleotide
gagaaacgcaaagggcagcg

SEQ ID NO: 8 - DNA, 20 bp, Artificial Sequence
  Description of Artificial Sequence Synthetic oligonucleotide
cggtccaccagccagtagca

SEQ ID NO: 9 - DNA, 20 bp, Artificial Sequence
  Description of Artificial Sequence Synthetic oligonucleotide
gtttactatcacggctccaa

SEQ ID NO: 10 - DNA, 20 bp, Artificial Sequence
  Description of Artificial Sequence Synthetic oligonucleotide
ggggagagcctcgctgccaa

SEQ ID NO: 11 - DNA, 20 bp, Artificial Sequence
  Description of Artificial Sequence Synthetic oligonucleotide
ccaaggccaagaacttgatg

SEQ ID NO: 12 - DNA, 20 bp, Artificial Sequence
  Description of Artificial Sequence Synthetic oligonucleotide
cacagtgggcattgatttca

SEQ ID NO: 13 - DNA, 20 bp, Artificial Sequence
  Description of Artificial Sequence Synthetic oligonucleotide
cgttaatggccgactggctc

SEQ ID NO: 14 - DNA, 20 bp, Artificial Sequence
  Description of Artificial Sequence Synthetic oligonucleotide
aatgccaagtggttaaagga

(19) United States
(12) Patent Application Publication
(10) Pub. No.: US 2021/0046109 A1
(43) Pub. Date: Feb. 18, 2021

(54) Suppressive Exosomes in Cancer and for Immunosuppression

(71) Applicant: The Regents of the University of California, Oakland, CA (US)

(72) Inventors: Robert Blelloch, San Francisco, CA (US); Mauro Poggio, San Francisco, CA (US); Tianyi Hu, San Francisco, CA (US)

(73) Assignee: The Regents of the University of California, Oakland, CA (US)

(21) Appl. No.: 16/980,391

(22) PCT Filed: Mar. 14, 2019
(86) PCT No.: PCT/US2019/022224

§ 371 Date: Sep. 12, 2020

Related U.S. Application Data
(60) Provisional application No. 62/790,464, filed on Jan. 9, 2019.
(60) Provisional application No. 62/643,163, filed on Mar. 14, 2018.

(51) Int. Cl. A61K 35/13; A61K 31/7105; A61P 35/00; C12N 5/09

(52) U.S. Cl. CPC A61K 35/13; C12N 5/0693; A61P 35/00; A61K 31/7105

(57) ABSTRACT

Suppressive extracellular vesicles (EVs) such as PD-L1-bearing exosomes are produced by cancer cells and promote systemic suppression of the immune system, enabling tumors to escape immune surveillance. Inhibitors of suppressive EVs reduce the suppressive activity and/or the production of suppressive EVs, relieving systemic immunosuppression, and may be use to increase the efficacy of a co-administered immunotherapy. Additionally, engineered cancer cells that have an impaired capacity to produce PD-L1-bearing exosomes can be administered to prime the immune system against resident tumors, overcoming the systemic suppression of the immune system by cancer cells. Also, exogenously produced PD-L1-bearing exosomes may be administered to a subject for the treatment of an immune-related condition or to promote therapeutic immunosuppression.

### CROSS-REFERENCE TO RELATED APPLICATIONS

This application is a 35 USC § 371 National Stage application of PCT International Application Number PCT/US2019/022224, entitled “Suppressive Exosomes in Cancer and for Immunosuppression,” filed Mar. 14, 2019, which claims the benefit of priority to U.S. Provisional Application Ser. No. 62/643,163, entitled “Inhibition of Cancer Derived Immunosuppressive Exosomes,” filed Mar. 14, 2018; and U.S. Provisional Patent Application Ser. No. 62/790,464, entitled “Exosomes in Cancer and Immunosuppressive Treatments,” filed Jan. 9, 2019, the contents which applications are hereby incorporated by reference.

The instant application contains a Sequence Listing which has been submitted electronically in ASCII format and is hereby incorporated by reference in its entirety. Said ASCII copy, created on Mar. 13, 2019, is named UCSF058PCT_SL.txt and is 3,601 bytes in size.

### STATEMENT REGARDING FEDERALLY SPONSORED RESEARCH OR DEVELOPMENT

This invention was made with government support under grant number U19CA179512 awarded by the National Institutes of Health. The government has certain rights in the invention.

### INTRODUCTION

Immunotherapy has revolutionized cancer therapy. Immune checkpoint protein inhibitors, such as antibodies against PD-L1 and PD-1, have shown effectiveness against a large number of cancer types including melanoma, non-small cell lung cancer, and renal cancer. This response includes durable remissions in many patients who had previously failed multiple other therapeutic strategies. However, even in these cancers, only ten to thirty percent of patients respond to anti-PD-L1/PD-1 therapy. In other cancers, such as prostate cancer, responses are rare. The basis of differential therapeutic success between patients and between cancers remains largely unknown.

PD-L1 is a membrane bound ligand found on the cell surface of many cell types and upregulated in the setting of inflammation or by a number of oncogenic lesions. It binds the PD-1 receptor on immune T cells leading to suppression of antigen-driven activation of T cells, keeping inflammatory responses in check. However, tumor cells can co-opt this mechanism to evade immune destruction. Therapeutic antibodies to PD-L1 and PD-1 block this interaction, which can then improve the anti-tumor immune response.

It is generally thought that PD-L1 tumor suppression of the immune system functions within the tumor bed, where cell surface PD-L1 is interacting directly with PD-1 on the surface of tumor infiltrating effector T cells. However, PD-L1 can also be found on surface of extracellular vesicles (EVs). Furthermore, EV PD-L1 levels have been associated with tumor progression in various contexts. For example, Theodoraki et al., 2018, Clinical significance of PD-L1⁺ exosomes in plasma of Head and Neck Cancer patients, Clin Cancer Res 24:896-905 demonstrated that PD-L1 levels on exosomes, correlate with disease progression in head and neck cancer patients, suggesting that circulating exosomal PD-L1 can act as an indicator of disease prognosis and immune activity in cancer patients. Yang et al., 2018, Exosomal PD-L1 harbors active defense function to suppress T cell killing of breast cancer cells and promote tumor growth Cell Res 28:862-864, suggested that exosomal PD-L1 suppresses T-cell killing of breast cancer cells. Chen et al., 2018, Exosomal PD-L1 contributes to immunosuppression and is associated with anti-PD-1 response. Nature 560, 382-386, showed that metastatic melanomas release PD-L1 exosomes and the abundance of such exosomes is predictive of anti-PD-1 therapy efficacy, suggesting that PD-L1 exosomes can systemically suppress the immune system. Ricklefs, et al., 2018, Immune evasion mediated by PD-L1 on glioblastoma-derived extracellular vesicles. Sci Adv 4, eaar2766, showed that glioblastoma PD-L1 EVs block T cell activation and proliferation and that an anti-PD-1 receptor blocking antibody significantly reversed the PD-L1 EV-mediated blockade of T cell activation.

These data show that PD-L1's role is neither limited to cell-cell interactions nor to the tumor microenvironment. Accordingly, there remains a need in the art for an improved understanding of the role of suppressive EVs in cancer. There also remains a need in the art for novel cancer therapies that can overcome suppressive effects of PD-L1 bearing exosomes and other suppressive EVs.

Relatedly, there is a need in the art for identifying subjects having substantial systemic immunosuppression by suppressive EVs such that appropriate treatment to relieve such immunosuppression may be directed to such subjects.

Relatedly, given the importance of circulating suppressive EVs in cancer biology and immunology, there is a need in the art for facile methods of quantifying suppressive exosome release.

Relatedly, given the suppressive power of PD-L1 bearing exosomes, there is a need in the art for novel applications of such EVs to therapeutically suppress the immune system.

### SUMMARY OF THE INVENTION

Disclosed herein are various inventions related to the immunosuppressive powers of circulating EVs bearing PD-L1 and other suppressive molecules.

In a first aspect, the scope of the invention is directed to inhibitors of suppressive EVs and uses thereof. As demonstrated herein, suppressive EVs such as PD-L1-bearing exosomes promote systemic suppression of the immune system, enabling tumors to escape immune surveillance. Inhibitors of suppressive EVs reduce the abundance of immune-suppressive agents, reinvigorating tumor surveillance. In one aspect, these inhibitors encompass agents that inhibit suppressive molecule production and/or packaging in EVs, such that the suppressive activity of EVs is reduced. In another aspect, these inhibitors encompass agents that inhibit the production and release of EVs, reducing the abundance of suppressive EVs in circulation. The invention further encompasses the novel use of suppressive EV inhibitors in the treatment of cancer with other co-administered immunotherapies. By relieving the systemic immunosuppression in a subject with suppressive EV inhibitors, the efficacy of a co-administered immunotherapy is increased.

In a second aspect, the scope of the invention is directed to novel methods of priming the T cells against cancer cells that evade immune attack by production of suppressive EVs. These methods employ novel compositions comprising engineered cancer cells that have an impaired capacity to produce suppressive EVs, such as PD-L1-bearing exosomes. In a first implementation, the engineered cancer cells comprise cancer cells with a reduced capacity to produce suppressive molecules such as PD-L1. In a second implementation, the engineered cancer cells comprise cancer cells with a reduced capacity to produce EVs, such as exosomes. For the treatment of cancer, the engineered cells are introduced to a cancer patient and elicit an immune response against resident tumors. By this method, the systemic suppression of the immune system is overcome and a robust immune response against cancer is promoted.

In a third aspect, the scope of the invention encompasses prognostic and diagnostic methods for identifying cancer patients for which the aforementioned treatments will be most efficacious. The methods encompass assessing the degree of systemic suppression in a subject. In a first implementation, the abundance of suppressive EVs such as PD-L1 exosomes is assessed. In an alternative implementation, the abundance of infiltrating immune cells in resident tumors is assessed. Those subjects found to have substantial systemic immune suppression are deemed highly suitable for treatments that inhibit or work around systemic immunosuppression mediated by suppressive EVs.

In a fourth aspect, the scope of the invention encompasses novel methods of quantifying suppressive exosome production. The scope of the invention encompasses fusion proteins comprising an EV associated molecule such as PD-L1 and a reporter moiety such as a fluorescent protein. Transformation of cancer cells with the fusion protein enables facile quantification and tracking throughout the body of suppressive exosomes produced by cancer cells.

In a fifth aspect, the scope of the invention encompasses therapeutic uses of suppressive exosomes. Cells are engineered to produce suppressive EVs such as PD-L1-bearing exosomes. These exogenously produced suppressive EVs may then be administered to subjects in order to promote systemic or localized immunosuppression, for example, to be used in the treatment of autoimmune conditions, to reduce inflammatory processes, and to prevent immune rejection of transplanted organs and other grafts.

### BRIEF DESCRIPTION OF THE DRAWINGS

FIG. 1. Schematic of in vitro functional assay of exosomal PDL1, depicting the interaction between T-cells, antigen-presenting cells, and PC-3 exosomes. MHC molecules on APCs bind antigens recognized by T-cell receptors, resulting in their activation. B7 and CD28 interact to enhance T-cell activation. Exosomal PD-L1 from PC-3 cells interacts with PD-1 on T-Cells to inhibit T-cell activation

FIGS. 2A and 2B. FIG. 2A depicts tumor growth following subcutaneous injection of 1*10⁶ WT, Rab27a null, or Pd-l1 null TRAMP cells into immunocompetent B6 mice. N=5 for each genotype. Error bars represent SEM. Tramp WT vs TRAMP Rab27a null, p<0.001. Tramp WT vs TRAMP Pd-l1 null, p<0.0001. Tramp WT vs TRAMP nSMase2 null, p<0.001 (Two-way ANOVA test). FIG. 2B depicts mouse survival curve in days following injection of cells. N=10 for each genotype. Tramp WT vs TRAMP Rab27a null, p<0.001. Tramp WT vs TRAMP Pd-l1 null, p<0.001. Tramp WT vs TRAMP nSMase2 null, p<0.001.

FIGS. 3A and 3B. FIG. 3A depicts a density trace for nanoparticle tracking of size and quantity of GFP⁺ vesicles from WT and Rab27a null PC3 cells expressing CD63-GFP. FIG. 3B depicts integration under curve for total particles, N=3.

FIG. 4 FIG. 4 depicts the quantification of PD-L1 in exosomes secreted by WT PC3, Rab27a null and nSMase2 null PC3 cells, N=6.

FIGS. 5A, 5B, 5C, 5D, 5E, 5F, 5G, 5H, 5I, 5J, 5K and 5L. FIG. 5A, 5B, 5C, 5D, 5E, 5F, 5G, 5H, 5I, 5J, 5K and 5L depict various measures in three sets of mice: Mice injected with wild type Tramp cells (WT), mice injected with Pd-l1 null TRAMP cells, and mice injected with Rab27a null TRAMP cells. N=5 mice/TRAMP genotype, 1*10⁶ cells injected per treatment. All measurements obtained 14 days post-injection, p<0.001 (Two-way ANOVA test). Error bars represent SEM. FIG. 5A depicts spleen weight in grams. FIG. 5B depicts flow cytometric quantification of the percent of CD8+ cells among CD45+, CD3+ cells in the draining lymph node. FIG. 5C depicts flow cytometric quantification of the percent of CD4+ cells among CD45+, CD3+ cells in the draining lymph node. FIG. 5D depicts flow cytometric quantification of the percent of regulatory T cells (T-reg) cells among CD45+, CD3+ cells in the draining lymph node. FIG. 5E depicts flow cytometric quantification of PD-1+ cells among CD8 T-cells in the draining lymph node. FIG. 5F depicts flow cytometric quantification of PD-1+ cells among CD4 T-cells in the draining lymph node. FIG. 5G depicts flow cytometric quantification of Tim3+ positive cells in CD8 T-cells in the draining lymph node. FIG. 5H depicts flow cytometric quantification of Tim3+ positive cells in CD4 T-cells in the draining lymph node. FIG. 5I depicts flow cytometric quantification of Granzyme B positive cells in CD8 T-cells in the draining lymph node. FIG. 5J depicts flow cytometric quantification of Granzyme B positive cells in CD4 T-cells in the draining lymph node.

FIG. 6. Tumor growth volume over time following secondary subcutaneous injection of 1*10⁶ WT TRAMP cells into immunocompetent B6 mice previously sham treated or injected with Pd-l1, Rab27a, or nSMase2 null TRAMP cells. N=5 for each condition. Tramp WT vs TRAMP WT pre-injected with TRAMP Rab27a null, p<0.001. Tramp WT vs TRAMP WT pre-injected with TRAMP Pd-l1 null, p<0.001. Tramp WT vs. TRAMP WT pre-injected with TRAMP nSMase2 null, p<0.001 (Two-way ANOVA test). Error bars represent SEM.

FIGS. 7A, 7B, and 7C. FIG. 7A depicts tumor growth over time following subcutaneous injection of 1*10⁶ WT, Rab27a null, or Pd-l1 null MC38 cells into immunocompetent B6 mice. N=5 for each genotype. Error bars represent SEM. MC38 WT vs MC38 Rab27a null, p<0.05. MC38 WT vs MC38 Pd-l1 null, p<0.05. MC38 WT vs MC38 Pd-l1 null; Rab27a null, p<0.05 (Two-way ANOVA test). FIG. 7B depicts mouse survival curve following injection of cells as in FIG. A. N=10 for each genotype. MC38 WT vs MC38 Rab27a null, p<0.001. MC38 WT vs MC38 Pd-l1 null, p<0.001. MC38 WT vs MC38 Pd-l1 null; Rab27a null, p<0.001 (Longrank test). FIG. 7C depicts the survival curve for mice injected with WT, Rab27a null, or Pd-l1 null MC38 cells followed by treatment with either anti-PD-L1 or isotype control antibody. N=5 for each condition. MC38 WT Isotype vs MC38 WT anti-PD-L1, p<0.01. MC38 Rab27a Isotype vs MC38 Rab27a anti-PD-L1, p<0.05. MC38 Pd-l1 Isotype vs MC38 Rab27a anti-PD-L1, N.S. (Longrank test).

FIGS. 8A, 8B, 8C, and 8D. FIG. 8A depicts TRAMP tumor growth in immunocompetent B6 mice that were singly injected with 1*10⁶ WT TRAMP cells or were co-injected with either Pd-l1 null or Rab27a null cells in one flank and with 1*10⁶ WT TRAMP cells in the other flank. N=5. Tramp WT vs TRAMP WT co-injected with TRAMP Rab27a null, p<0.001. Tramp WT vs TRAMP WT co-injected with TRAMP Pd-l1 null, p<0.001. Tramp WT vs TRAMP WT co-injected with TRAMP nSMase2 null, p<0.01 (Two-way ANOVA test). FIG. 8B depicts tumor growth of Pd-l1 null or Rab27a null TRAMP cells in mice singly injected or co-injected with WT TRAMP cells. N=5. Error bars represent SEM. FIG. 8C depicts mouse survival curve mice singly injected with WT TRAMP cells or doubly injected with WT and Pd-l1 null or Rab27a null cells. N=5 for each genotype. FIG. 8D depicts scoring of Histological analysis of lymphocyte infiltration of tumors under the noted conditions. Lymphocyte infiltration of tumors for each mouse was rated as severe, moderate, mild, or none.

FIG. 9A and FIG. 9B. FIG. 9A depicts tumor growth over time following subcutaneous injection of 1*10⁶ MC38Rab27a null cells followed by tail vein exosomes derived from MC38 WT and MC38 Pd-l1 null cells grown in vitro. MC38 Rab27a null treated with WT vs. Pd-l1 null exosomes, p<0.01 (Two-way ANOVA test). FIG. 9B depicts the survival curve following injection of as in 9A. MC38 Rab27a null tumors treated with WT vs. Pd-l1 null exosomes, p<0.0 Error bars represent SEM.

FIG. 10. FIG. 10 depicts a Summary Schematic. Left: In absences of exosomes, anti-tumor T cells are activated in draining lymph node leading to systemic immunity and memory, even towards tumor cells secreting exosomal PD-L1. Middle: In presence of exosomes carrying PD-L1, T cell response is suppressed in draining lymph node enabling tumor growth. Right: In presence of exosomes lacking PD-L1, anti-tumor T cells are activated in draining lymph node, leading to systemic immunity and memory, even towards tumor cells secreting exosomal PD-L1.

FIGS. 11A and 11B. FIG. 11A depicts wild type TRAMP tumor size over time. WT TRAMP cells were injected in one flank and allowed to to grow for 35 days, followed by injection of Rab27a-, sNmase2-, or PD-L1-mutant cells. FIG. 11B depicts survival curves for the different treatments.

FIGS. 12A 12B, 12C, 12D, 12E, 12F, 12G, 12H, 12I, and 12J. FIG. 12A 12B, 12C, 12D, 12E, 12F, 12G, 12H, 12I, and 12J depict the effects of exogenously introduced exosomal PD-L1. Mice were transplanted with 1*10⁶ Rab27a null TRAMP cells, followed by three times per week tail vein injections of exosomes that were collected from either WT or Pd-l1 null TRAMP cells grown in vitro. All measurements were performed at 14 days. Each dot represents an individual mouse. Error bars represent mean and SD. *p<0.05, **p<0.01, ***p<0.001 (Student T-test). Error bars represent SEM. FIG. 12A depicts spleen weight in grams. FIG. 12B depicts flow cytometric quantification of the percent of CD8+ cells among CD45+, CD3+ cells in the draining lymph node. FIG. 12C depicts flow cytometric quantification of the percent of CD4+ cells among CD45+, CD3+ cells in the draining lymph node. FIG. 12D depicts flow cytometric quantification of the percent of regulatory T cells (T-reg) cells among CD45+, CD3+ cells in the draining lymph node. FIG. 12E depicts flow cytometric quantification of PD-1+ cells among CD8 T-cells in the draining lymph node. FIG. 12F depicts flow cytometric quantification of PD-1+ cells among CD4 T-cells in the draining lymph node. FIG. 12G depicts flow cytometric quantification of Tim3+ positive cells in CD8 T-cells in the draining lymph node. FIG. 12H depicts flow cytometric quantification of Tim3+ positive cells in CD4 T-cells in the draining lymph node. FIG. 12I depicts flow cytometric quantification of Granzyme B positive cells in CD8 T-cells in the draining lymph node. FIG. 12J depicts flow cytometric quantification of Granzyme B positive cells in CD4 T-cells in the draining lymph node

### DETAILED DESCRIPTION OF THE INVENTION

The various inventions described herein may be applied in the treatment of cancer in a subject. “Cancer,” as used herein, will refer to any neoplastic condition. For example, the neoplastic condition may comprise a cancer selected from the group consisting of the following: bladder cancer, brain cancer, breast cancer, cervical cancer, colorectal cancer, esophageal cancer, head and neck cancer, kidney cancer, lung cancer, leukemia, lymphoma, myeloma, multiple melanoma, ovarian cancer, pancreatic cancer, prostate cancer, sarcoma, and skin cancer.

The subject may be a human subject, e.g. a cancer patient. The subject may alternatively be a non-human animal such as a mouse, rat, dog, cat, rodent, or any other animal species, including test animals, cancer models, and veterinary subjects.

Certain embodiments of the inventions disclosed herein are directed to the treatment of a condition. “Treatment” as used herein will encompass any therapeutic action or objective, for example, reducing the symptoms or severity of a condition, preventing the condition, slowing the progression of the condition, etc. In the context of cancer, treatment may encompass a reduction in the number of cancer cells, a reduction in tumor size, reductions in the metastatic potential or activity of cancer cells, arresting the growth or spread of tumors, arresting the progression of precancerous cells to cancer or the progression of cancer to more advanced stages.

Certain embodiments are directed to extracellular vesicles (EVs). As used herein, EVs will refer to any extracellular vesicle, for example, exosomes and microvesicles. Exosomes are produced endosomally, accumulated within multivesicular bodies during biogenesis and released upon exocytosis of the multivesicular bodies. Exosomes are generally in the size range of 30-120 nm. Microvesicles are produced from the outward budding and fission of the plasma membrane, and may range in size from 20 nm up to 1000 nm or greater

Certain aspects of the invention encompass systemic suppression of the immune system. Systemic suppression includes any inhibition or downregulation of specific and/or general immune processes, such as the activity, activation of, or interaction between of selected immune cells. In the context of EVs, systemic suppression of the immune system encompasses suppressive action distal to the source of the EVs or site of EV introduction. In one implementation, the invention encompasses methods of inhibiting systemic immunosuppression mediated by cancer cells by inhibition of immune-suppressive exosomes.

Certain aspects of the invention encompass the use of therapeutically effective amounts of administered agents. A therapeutically effective amount is an amount sufficient to cause a measureable biological response and/or any therapeutic effect.

As used herein, “a” or “an” may mean one or more, unless clearly indicated otherwise. As used in the specification and the claim(s), when used in conjunction with the word “comprising,” “including,” “containing,” or “having,” or any variation of these terms, the words “a” or “an” may mean one or more than one.

Inhibition of Suppressive EVs to Treat Cancer. The research findings presented herein demonstrate that cancer cells can suppress immune responses against tumors by the secretion of exosomes bearing immunosuppressive molecules. The result is a systemic suppressive effect on immune components distal to the tumor. For example, as demonstrated herein, exosome-derived PD-L1 from prostate tumors can inhibit T cell priming in lymph nodes, remote from the tumor, ultimately reducing T cell migration to the tumor site. As demonstrated herein, inhibition of suppressive EVs provides a novel strategy for the treatment of cancer.

The scope of the invention encompasses the use of various suppressive EV inhibitors. As used herein, a “suppressive EV inhibitor” comprises any composition of matter which inhibits the suppressive activity of suppressive EVs, or which inhibits the production, secretion, or abundance of suppressive EVs in a subject. In certain aspects, the suppressive EV inhibitors will be used to relieve systemic immunosuppression. In this context, “systemic immunosuppression” means any immunosuppressive effect distal to a tumor, for example, a general, non-specific reduction in immune activity or, alternatively any reduction in the priming of immune cells such as T cells, B cells, NK cells and/or neutrophils against specific tumor antigens and/or the migration of activated cells to the tumor.

In a first aspect, the scope of the invention encompasses a method of inhibiting systemic suppression of the immune system by suppressive EVs in a subject by the administration to the subject of a therapeutically effective amount of an of a suppressive EV inhibitor. In a related aspect, the scope of the invention encompasses a suppressive EV inhibitor for use in inhibiting systemic suppression of the immune system by suppressive EVs. In one aspect, the scope of the invention encompasses the use of a suppressive EV inhibitor in the manufacture of a medicament for the treatment of cancer.

In another aspect, the scope of the invention encompasses a method of treating cancer in a subject by the administration to the subject of a therapeutically effective amount of an of a suppressive EV inhibitor. In a related aspect, the scope of the invention encompasses a suppressive EV inhibitor for use in the treatment of cancer.

In another aspect, relief of systemic immunosuppression by suppressive EV inhibitors is used to enhance the efficacy of a co-administered immunotherapy treatment. In one aspect, the scope of the invention encompasses a method of enhancing the efficacy of an immunotherapy treatment in a subject receiving such treatment by the administration to the subject of a therapeutically effective amount of a suppressive EV inhibitor. In a related aspect, the scope of the invention encompasses a suppressive EV inhibitor for use in enhancing the efficacy of an immunotherapy treatment.

The suppressive EV inhibitor may comprise various compositions of matter that act by any number of mechanisms. In a first implementation, the inhibitor of suppressive EVs inhibits the suppressive capacity of suppressive EVs. For example, the suppressive EV inhibitor may inhibit the capacity of suppressive EVs to downregulate activation of T cells. For example, in one embodiment, the suppressive EV inhibitor reduces the capacity of suppressive EVs, for example, exosomes, comprising PD-L1 to interact with with PD-1 on the surface of T cells or within T cells, alone or in cooperation with other cells (i.e. macrophages or antigen presenting cells).

In one embodiment, the inhibitor of EV suppressive activity acts by inhibiting the suppressive activity, i.e., signaling functions, of suppressive EVs. In one embodiment, the inhibitor of EV suppressive activity disrupts the interaction of one or more suppressive molecules present on or within suppressive EVs with receptors on immune cells. In one embodiment, the one or more suppressive molecules comprises PD-L1 and the inhibitor of suppressive activity interferes with the interaction of EV-borne PD-L1 with the PD-1 receptor on or within immune T cells.

In one embodiment, the inhibitor of suppressive EV activity comprises a small molecule. In one embodiment, the small molecule disrupts PD-L1 interactions with immune cells. Exemplary small molecules include those describe in United States Patent Application Publication Number 20190016681, entitled “Inhibitors of the pd-1/pd-11 protein/protein interaction,” by Domling; PCT International Patent Application Publication Number WO 2015034820, entitled “Compounds useful as immunomodulators,” by Chupak and Zheng; and PCT International Patent Application Publication Number WO 2015160641, entitled “Compounds useful as immunomodulators,” by Chupak et al.

In one embodiment, the inhibitor of suppressive EVs comprises a peptide that disrupts or inhibits suppressive EV activity. In one embodiment, the peptide comprises an inhibitor of PD-L1 interaction with immune cells. Exemplary peptides include peptides described in United States Patent Application Publication Number 20150294898, entitled “Macrocyclic inhibitors of the pd-1/pd-11 and cd80(b7-1)/pd-11 protein/protein interactions,” by Miller et al. In one embodiment, the peptide comprises an antibody or fragment thereof which disrupts the interaction between EV suppressive molecules and immune cells. In one embodiment, the antibody or fragment thereof comprises a composition of matter which binds to PD-L1, PD-1, or which otherwise disrupts PD-L1-PD-1 interactions. Exemplary antibodies or fragments thereof include antibodies and antigen binding regions described in United States Patent Application Publication Number 20180244781, entitled “Pd-1/pd-11 inhibitors for the treatment of cancer,” by Cuillerot et al.; United States Patent Application Publication Number, 8,217,149, entitled “Anti-PD-L1 antibodies, compositions and articles of manufacture,” by Irving et al.; U.S. Pat. No. 8,552,154, entitled “Anti-PD-L1 antibodies and uses therefor,” by Freeman et al.

In another embodiment, the inhibitor of EV suppressive activity comprises a construct which disrupts the expression of one or more suppressive molecules present in suppressive EVs. In one embodiment, the suppressive molecules is PD-L1. In one embodiment, the suppressive EV inhibitor reduces the abundance of or substantially eliminates PD-L1 in secreted EVs.

In alternative embodiments the one or more suppressive molecules is selected from the group consisting of PD-1, PD-2, adenosine A2A receptor, B7-H3 (CD276), B7-H4 (VTCN1), BTLA, CTLA-4, Indoleamine 2,3-dioxygenase, Killer-cell Immunoglobulin-like Receptor, Lymphocyte Activation Gene-3,NOX-2, PD-1, TIM-3, or V-domain Ig suppressor of T cell activation.

Exemplary agents for disrupting the expression of suppressive molecules include short interfering RNAs, hairpin RNAs, zinc finger nucleases, transcription activator-like effector nucleases, or CRISPR systems (which will be understood herein to include CRISPR/Cas9, CRISPR/dCAS9-KRAB, and other CRISPR-based systems for genetic manipulation), or other compositions of matter which can selectively disrupt the expression of a target gene. In one embodiment, the suppressive EV inhibitor comprises an agent that selectively targets suppressive molecules for degradation, for example, a proteolysis-targeting chimera, or PROTAC.

In one embodiment, the inhibitor of suppressive EVs acts by inhibiting the packaging of suppressive molecules in EVs. The result will be the production of EVs that are not suppressive, or having a lower suppressive activity, by the reduction in numbers of suppressive molecules. In one embodiment, the inhibitor of suppressive EVs inhibits the loading of PD-L1 in exosomes. In one embodiment, the inhibitor of suppressive EVs acts by inhibiting the expression of one or more genes implicated in the loading of suppressive molecules, e.g. a gene involved in PD-L1 loading in exosomes.

In a second implementation, the inhibitor of suppressive EVs acts by inhibiting the production of EVs. In one embodiment, the inhibitor of suppressive EVs acts by inhibiting the production of exosomes. In one embodiment, the inhibitor of suppressive EVs acts by inhibiting the production of microvesicles. In one embodiment, the inhibitor of suppressive EVs comprises a composition of matter which inhibits the expression, abundance, and/or activity of proteins expressed by an EV production gene, i.e. a gene wherein the expression of which is involved in or necessary for the production, secretion, uptake and/or persistence of EVs, e.g., exosomes. In one embodiment, the EV production gene is a Rab gene. In one embodiment, the EV production gene is Rab27, including either Rab27a or Rab27b, which is involved in the fusion of the multivesicular bodies to the plasma membrane, facilitating exosome release. In one embodiment, the EV production gene is gene coding for an endosomal sorting complex required for transport (ESCRT) element, including, for example, a gene coding for a protein of the ESCRT0, ESCRT1, ESCRT2, and ESCRT3 complexes. In one embodiment, the EV production gene is nSMase2, which promotes budding of intravesicular vesicles. In one embodiment, the EV production gene is any of the genes coding for proteins of the Soluble N-ethylmaleimide-sensitive factor attachment protein receptor (SNARE) complexes. Exemplary EV biogenesis further include, for example, ALIX, TSG101, HRS, syntenin, ubiquitin, clathrin, VPS32, VPS, SNAP23, VAMP3, VAMP7, YKT6, RAB-11, RAB-35, RAB-5, RAB-7, and other genes, for example, as described in Neil et al. 2018. Nat Rev Mol Cell Biolo. 19:213-228.

In one embodiment, the suppressive EV inhibitor comprises a small molecule which disrupts the production of EVs, e.g. exosomes. In one embodiment, the small molecule is an inhibitor of Rab27a. In one embodiment, the inhibitor of Rab27a is Nexinhib20. In one embodiment, the small molecule is an inhibitor of nSMase2. In one embodiment, the inhibitor is cambinol, GW4869, or 2,6-Dimethoxy-4-(5-Phenyl-4-Thiophen-2-yl-1H-Imidazol-2-yl)-Phenol (DPTIP). Other exemplary small molecule inhibitors of EV biogenesis include tipifarnib, neticonazole, climbazole, isoproterenol, ketoconazole, mitotane, triademenol, pentetrazol, Cannabidiol, simvastatin, Brefeldin A, tunicamycin, dimethyl amiloride, Monensin, chloramidine, and bisindolylmaleimide-I. Additional inhibitors of nSMase2 include those described in PCT International Patent Application Publication Number WO2018129405, entitled SMALL MOLECULE INHIBITORS OF NEUTRAL SPHINGOMYELINASE 2 (nSMase2) FOR THE TREATMENT OF NEURODEGENERATIVE DISEASES, by Slusher et al.; PCT International Patent Application Publication Number WO 02/06644, entitled “4H-1,2,4-TRIAZOLE-3(2H)-THIONE DERATIVES AS SPHINGOMYELINASE INHIBITORS,” by Delaet et al.; and PCT International Patent Application Publication Number WO 02/066443, entitled “2-THIOXO-1,2,3,4-TETRAHYDROPYRIMIDINE DERIVATIVES,” by Wilson et al. It will be understood that the use of analogs and derivatives of the foregoing listed compounds is within the scope of the invention.

In one embodiment, the suppressive EV inhibitor comprises a peptide which disrupts the biogenesis and secretion of exosomes. Exemplary peptides include those described in United States Patent Application Publication Number 20180305412, entitled “Compositions and methods for treating diseases by inhibiting exosome release,” by Bond et al. In one embodiment, the peptide is an antibody or antibody fragment directed to the expression product of an exosome production gene, for example an intrabody.

In one embodiment, the suppressive EV inhibitor comprises an agent which disrupts the expression of one or more exosome biogenesis genes. For example, in various embodiments, the inhibitor may comprise a short interfering RNA, a hairpin RNA, a zinc finger nuclease, a transcription activator-like effector nuclease, or a CRISPR system. In one embodiment, the suppressive EV inhibitor comprises an agent which disrupts the expression of Rab27a. In one embodiment, the suppressive EV inhibitor comprises an agent which disrupts the expression of nSMase2.

In one embodiment, the suppressive EV inhibitor comprises an agent that selectively targets for degradation proteins that facilitate EV production, for example, a PROTAC or like construct. In one embodiment, the targeted protein is Rab27a. In one embodiment the targeted protein is nSMase2.

Exemplary inhibitors of suppressive EVs include the following. In one embodiment, the inhibitor of suppressive exosomes is an siRNA construct which reduces the expression of Rab27a, for example SEQ ID NO: 1: CUGUUAUGUAGAACGCUGA (Dharmacon, Inc); or SEQ ID NO: 2: GCUGCAGCUUUGUAUGAUU (Dharmacon, Inc.). In one embodiment, the inhibitor of suppressive exosomes is a CRISPR/Cas9 construct comprising an sgRNA, for example: SEQ ID NO: 3, human Pd-l1 sgRNA guide sequence GGTTCCCAAGGACCTATATG (Elim Biopharma, Inc.); SEQ ID NO: 4, human Pd-l1 sgRNA guide sequence ACAGAGGGCCCGGCTGTTGA (Elim Biopharma, Inc.); SEQ ID NO: 5, human Rab27a sgRNA guide sequence CCAAAGCTAAAAACTTGATG (Elim Biopharma, Inc.); SEQ ID NO: 6, human Rab27a sgRNA guide sequence CAACAGTGGGCATTGATTTC (Elim Biopharma, Inc.); SEQ ID NO: 7, Human nSMase2 sgRNA guide sequence GAGAAACGCAAAGGGCAGCG (Elim Biopharma, Inc.); SEQ ID NO: 8, Human nSMase2 sgRNA guide sequence CGGTCCACCAGCCAGTAGCA (Elim Biopharma, Inc.); SEQ ID NO: 9, mouse Pd-l1 sgRNA guide sequence GTTTACTATCACGGCTCCAA (Elim Biopharma, Inc.); SEQ ID NO: 10, mouse Pd-l1 sgRNA guide sequence GGGGAGAGCCTCGCTGCCAA (Elim Biopharma, Inc.); SEQ ID NO: 11, mouse Rab27a sgRNA guide sequence CCAAGGCCAAGAACTTGATG (Elim Biopharma, Inc.); SEQ ID NO: 12, mouse Rab27a sgRNA guide sequence CACAGTGGGCATTGATTTCA (Elim Biopharma, Inc.); SEQ ID NO: 13, mouse nSMase2 sgRNA guide sequence CGTTAATGGCCGACTGGCTC (Elim Biopharma, Inc.); and SEQ ID NO: 14, Mouse nSMase2 sgRNA guide sequence AATGCCAAGTGGTTAAAGGA (Elim Biopharma, Inc.).

The suppressive EV inhibitors of the invention may be delivered to tumor cells by any suitable means known in the art. In one embodiment, the scope of the invention encompasses a composition of matter comprising a suppressive EV inhibitor and a delivery agent. Exemplary delivery agents include viral and non-viral systems. Viral platforms are useful for polynucleotide-based suppressive EV inhibitors and may also be used in the delivery of other agents. Exemplary viral delivery agents include adenoviruses, vaccinia viruses, adeno-associated viruses, retroviruses, and herpes virus. Non-viral agent delivery systems include nanomaterials such as biomaterials or nanoparticles for the delivery of agents. Exemplary delivery agents include antibodies, intrabodies, lipids, polymers, graphene, carbon nanotubes, nanospheres, mesoporous nanoparticles, dendrimers, cationic liposomes, and other delivery compositions known in the art. Delivery of the suppressive EV inhibitor may be targeted to tumor cells by ligands and other recognition elements on the selected delivery agent, for example, targeting species that are uniquely or preferentially expressed on tumor cells, for example, integrins, receptors, and other species present in the cell membranes of cancer cells. Delivery of the suppressive EV inhibitor may be accomplished by passive means, for example, accumulation of agents delivered in the circulatory system due to the enhanced permeability and retention of tumor blood vessels. Alternatively, agent delivery may be actively assisted by biolistic methods, electroporation, sonoporation, magnetofection, and chemical transfection agents.

Exemplary embodiments of the invention include a delivery agent in combination with a suppressive EV inhibitor that reduces the expression of a suppressive molecule or that reduces the expression of an EV production gene. For example, the suppressive EV inhibitor may comprise a construct that reduces the expression of PL-D1 and/or Rab27a and/or nSMase2. The suppressive EV inhibitor may comprise an a short interfering RNA, a hairpin RNA, a zinc finger nuclease, a transcription activator-like effector nucleases, or a CRISPR system, in combination with a delivery agent comprising a viral or non-viral nanoparticle configured to deliver the suppressive EV inhibitor to cancer cells in vivo or in vitro, for example, for use in the treatment of cancer.

In a first implementation, the suppressive EV inhibitor is administered as a standalone treatment. In this implementation, the administration of the agent inhibits the suppressive capacity or abundance of suppressive EVs, e.g., PD-L1-bearing exosomes. This results in the relief of systemic immunosuppression by suppressive EVs and enables native immune responses in the subject to more effectively target and destroy cancer cells.

In a second implementation, the suppressive EV inhibitor of the invention is co-administered with one or more immunotherapy agents. The efficacy of immunotherapies is uneven, being very effective for some subjects, and less effective or wholly ineffective for others. It is believed by the inventors of the present disclosure that, in many cases, immunotherapies are ineffective because of the systemic immunosuppressive effects of suppressive EVs produced by tumors. Accordingly, co-administration of a suppressive EV inhibitor with an immunotherapy agent will provide relief of systemic immunosuppression and will enhance the efficacy of the co-administered immunotherapy.

In one embodiment, the suppressive EV inhibitor is co-administered with an immune checkpoint inhibitor. The immune checkpoint inhibitor may comprise any immune checkpoint inhibitor known in the art. Exemplary immune checkpoint inhibitors include, for example, inhibitors of CTLA-4, for example, Ipilimumab; inhibitors of PD-1, for example, Nivolumab and Pembrolizumab; and inhibitors of PD-L1, for example Atezolizumab, Avelumab, and Durvalumab.

In one embodiment, the suppressive EV inhibitor is co-administered with a cellular immunotherapy agent. In one embodiment, the suppressive EV inhibitor is co-administered with dendritic cells that have been primed ex-vivo (e.g. Sipuleucel-T). In one embodiment, the suppressive EV inhibitor is co-administered with chimeric antigen receptor T-cells (e.g., Tsagenlecleucel and axicabtagene ciloleucel). In one embodiment, the suppressive EV inhibitor is co-administered with a tumor-infiltrating lymphocyte primed ex-vivo to recognize tumor antigens.

In one embodiment, the suppressive EV inhibitor is co-administered with an immunotherapy comprising an agent which primes immune cells in vivo. For example, the agent may comprise a viral construct which targets tumor cells to express immunogenic factors (e.g. the cytokine GM-CSF), a tumor cell lysate, or an antigen-bearing antibody targeted to dendritic cells (e.g. targeted to TLR3, TLR7, TLR8, or CD40).

In one embodiment, the suppressive EV inhibitor is co-administered with an immunotherapy comprising a cytokine. For example, the cytokine may comprise interferon-alpha, interleukin-2, or GM-CSF.

In one embodiment, the suppressive EV inhibitor is co-administered with an immunotherapy agent comprising an antibody directed to a cancer-associated antigen. Such therapeutic antibodies bind epitopes that are overexpressed or exclusively present on tumor cells and facilitate cytotoxic immune responses, for example by antibody-dependent cell-mediated cytotoxicity (ADCC) or complement-dependent cytotoxicity. Exemplary anti-cancer antibodies include antibodies against CD20 (e.g., Ofatumumab, Rituximab) and antibodies against CD52 (e.g., Alemtuzumab). Other anti-cancer antibodies downregulate aberrant cell growth or otherwise attenuate cancer, for example, antibodies against the HER2 pathway (e.g. Trastuzumab). Anti-cancer antibodies further encompass antibody-drug conjugates, for example Trastuzumab entamsine, Gemtuzumab ozogamicin, Brentuximab vedotin, and Inotuzumab ozogamicin.

As above, the scope of the invention encompasses a suppressive EV inhibitor for use in enhancing the efficacy of an immunotherapy, for example, wherein the immunotherapy is selected from the group consisting of a checkpoint inhibitor, a primed dendritic cell, a tumor-infiltrating lymphocyte, a chimeric antigen receptor T-Cell, an agent for the priming of immune cells in vivo, and a cytokine. In one embodiment, the suppressive EV inhibitor is an inhibitor of Rab27a. In one embodiment, the suppressive EV inhibitor is an inhibitor of nSMase2. In one embodiment, the suppressive EV inhibitor is an inhibitor of PD-L1 expression or packaging in EVs.

In other implementations, the suppressive EV inhibitor is co-administered with a cancer fighting agent other than an immunotherapy agent. For example, anti-cancer agents such as chemotherapeutic drugs or kinase inhibitors may be co-administered with the suppressive EV inhibitor.

In certain cases, the suppressive EV inhibitor and the co-administered agents are packaged and distributed as a combination product. For example, the two agents may be packaged together e.g. in two separate compartments or containers (e.g. vials) within a common packaging element. In some embodiments, EV inhibitor and the one or more co-administered agents are compatible for combination in a single formulation that can be injected, infused, or otherwise delivered.

Co-administration, as used herein, means any temporal overlap or temporal proximity between two treatments. Co-administration encompasses any administration schedule wherein the suppressive EV inhibitor and one or more additional anti-cancer agents are administered such that the relief of systemic immune suppression by the suppressive EV inhibitor improves the efficacy of the co-administered treatment. In one embodiment, the suppressive EV inhibitor is administered as a series of dosages over a first time interval, and the one or more co-administered agents is administered as a series of individual dosages administered over a second time interval, wherein the first and second time intervals are wholly or partially overlapping. Alternatively, the suppressive EV inhibitor and co-administered agent are administered in non-overlapping time frames, but within sufficient temporal proximity that the suppressive EV inhibitor enhances the efficacy of the co-administered agent. In some implementations, the suppressive EV inhibitor is administered prior to and/or concurrently with the administration one or more co-administered agents.

The suppressive EV inhibitors may be administered by any drug delivery method appropriate for the selected agent. The suppressive EV inhibitors may be administered by intravenous injection, infusion, intracorporeal injection, intratumoral injection, oral ingestion, suppository, inhalation, or topically, as suitable for the selected agent and the selected formulation. The suppressive EV inhibitor may be selectively delivered to tumor cells by targeted viral vectors, antibodies, and other targeting technologies known in the art. The suppressive EV inhibitor may be formulated with suitable carriers, excipients, buffers, preservatives and other suitable compositions for efficacious delivery, as known in the art.

The suppressive EV inhibitor will be administered in a therapeutically effective amount, i.e., an amount sufficient to induce a measurable physiological or therapeutic effect, e.g. inhibition of the suppressive activity or abundance of suppressive EVs.

Engineered Cancer Cell for Immunization Against Tumors. The scope of the invention encompasses various methods of treating cancer by stimulation of the immune system with cancer cells lacking the ability to produce suppressive EVs, such as PD-L1-bearing exosomes. This methodology is based on the discovery that PD-L1 bearing exosomes play a powerful role in suppressing host immune response against tumors. Critically, as demonstrated herein, this suppression may be overcome by presenting the immune system with cancer cells lacking the capacity to produce suppressive EVs.

In one aspect, the scope of the invention encompasses a method of treating cancer by the following general method:
cancer cells are obtained from a subject having a tumor or other neoplastic condition;
the cancer cells are engineered to reduce suppressive EV activity or production; and
the engineered cancer cells are introduced into the subject directly or via a medical device (i.e. encapsulated in a biocompatible membrane), wherein the host immune system response to the engineered cells provides an immunotherapeutic response to like, resident cancer cells.

In a related aspect, the scope of the invention encompasses cancer cells engineered to have a reduced capacity for systemic immunosuppression, for use in the treatment of cancer. Such cells may comprise cells wherein the EVs they produce have a reduced capacity for immunosuppression relative to EVs produced by like, non-engineered cells. Such cells may comprise cells that are deficient in the production of EVs, relative to like non-engineered cells. In one embodiment, the scope of the invention encompasses the use of cancer cells engineered to have a reduced capacity for systemic immunosuppression in the manufacture of a medicament for the treatment of cancer.

The engineered cancer cells of the invention may comprise any cell derived from a tumor. In one embodiment, the engineered cancer cell is an autologous cancer cell isolated from a subject suffering from cancer. Advantageously, such cells will have an identical immunologic profile to the host cancer cells from which they are derived, provoking an effective and specific immune response against the source tumors, and being immunologically compatible with the host subject. The cells may comprise cells obtained from a biopsy, for example, a needle biopsy, surgical biopsy, bone marrow biopsy, skin biopsy, or other technique by which cancerous cells or tissue is obtained or extracted. Alternatively, cells may be obtained from a surgical resection procedure wherein a tumor is removed from the subject. Alternatively, the cells may be circulating cells, e.g. leukemia cells or metastatic cells isolated from a blood, lymph, or tissue sample.

Cells extracted from the subject may be directly engineered or may be propagated prior to engineering. Culture of cancer cells may be by methods known in the art, for example by the use of suitable culture media, vessels, and cell culture techniques. For example, tumor tissue removed from the subject may be dissociated, sorted, and plated on growth medium or placed in suspension culture, by methods known in the art.

In an alternative implementation, the engineered cancer cell of the invention is an allogenic cancer cell, i.e. a cancer cell derived from a different subject than the subject to be treated, but having an immunologic phenotype characteristic of the type of cancer being treated, so as to be capable of invoking an immune response against cancer cells resident in the subject.

In one embodiment, the scope of the invention encompasses an engineered cancer cell wherein EVs produced by the cell have a reduced abundance of suppressive molecules, relative to like, non-engineered cells, for use in the treatment of cancer. In one embodiment, the cancer cell is a cancer cell that produces PD-L1 bearing exosomes that has been engineered to produce less PD-L1 in exosomes than is produced by like non-engineered cells. The abundance of suppressive molecules may be assessed by any suitable measure, including, for example, the number of suppressive molecules per EV or per EV mass, the mass of suppressive molecules produced per EV or per EV mass, the suppressive activity of the produced EVs, or any other measure of suppressive molecule abundance. The reduced abundance of suppressive molecules may be any substantial reduction in suppressive molecule abundance, for example a reduction of at least 5%, at least 10%, at least 15%, at least 20%, at least 30%, at least 40%, at least 50%, at least 60%, at least 70%, at least 80%, at least 90%, at least 95% reduction relative to like non-engineered cells. Such reduction may be assessed by comparing the average abundance or activity of suppressive molecules present in a representative sample of engineered cells to the average abundance or activity of suppressive molecules in a representative set of non-engineered cancer cells.

In a first implementation, the reduction in abundance of suppressive molecules is achieved by disrupting the expression of one or more selected suppressive molecules. In a primary embodiment, the engineered cancer cell is derived from cancer cells that produces PD-L1-bearing exosomes and the engineered cell produces exosomes with less PD-L1 molecules per exosome than its non-engineered source cells, for example, by disruption of PD-L1 expression. In alternative embodiments, the one or more selected suppressive molecules is selected from the group consisting of PD-1, PD-2, adenosine A2A receptor, B7-H3 (CD276), B7-H4 (VTCN1), BTLA, CTLA-4, Indoleamine 2,3-dioxygenase, Killer-cell Immunoglobulin-like Receptor, LAG-3 (Lymphocyte Activation Gene-3),NOX-2, PD-1, TIM-3, or V-domain Ig suppressor of T cell activation

In alternative embodiments, the abundance of one or more selected suppressive molecules is achieved by disrupting processes integral to the packaging of the selected molecules in secreted EVs, for example, the transport and integration of PD-L1 molecules in exosomes.

In one embodiment, the scope of the invention encompasses an engineered cancer cell having a reduced capacity to produce suppressive EVs, relative to that of like, non-engineered cells, for use in the treatment of cancer. In one embodiment, the cancer cell is a cancer cell that is engineered to produce less exosomes than are produced by like non-engineered cells. The production of EVs may be assessed by any suitable measure, including, for example, the numbers of EVs produced, the mass of EVs produced, the rate of EV production, or other measures of EV production. The reduced production of suppressive EVs may be any substantial reduction in suppressive EV production, for example a reduction of at least 5%, at least 10%, at least 15%, at least 20%, at least 30%, at least 40%, at least 50%, at least 60%, at least 70%, at least 80%, at least 90%, at least 95% reduction relative to like non-engineered cells. Such reduction may be assessed by comparing the average production of suppressive EVs in a representative sample of engineered cells to the average production of suppressive EVs in a representative set of like, non-engineered cancer cells.

In a first implementation, the reduction in suppressive EV production is achieved by disrupting the expression of one or more EV production gene, i.e., a gene involved in or necessary for EV biogenesis, release, or secretion. In one embodiment, the EV production gene is a Rab gene. In one embodiment, the EV production gene is Rab27a. In one embodiment, the EV production gene is gene coding for an endosomal sorting complex required for transport (ESCRT) element, including, for example, any gene coding for a protein of the ESCRT0, ESCRT1, ESCRT2, and ESCRT3 complexes. In one embodiment, the EV production gene is nSMase2. In one embodiment, the EV production gene is any of the genes coding for proteins of the Soluble N-ethylmaleimide-sensitive factor attachment protein receptor (SNARE) complexes. Exemplary EV biogenesis genes include, for example, ALIX, TSG101, HRS, syntenin, ubiquitin, clathrin, VPS32, VPS, SNAP23, VAMP3, VAMP7, YKT6, RAB-11, RAB-35, RAB-5, RAB-7, and other genes, for example, as described in Neil et al. 2018. Nat Rev Mol Cell Biolo. 19:213-228

In a primary embodiment, the engineered cancer cell is derived from cancer cells that produce PD-L1-bearing exosomes and the engineered cell produces less PD-L1-bearing exosomes than its non-engineered source cells. In one embodiment, the engineered cancer cell that produces less PD-L1 bearing exosomes comprises a cancer cell wherein Rab27a expression or activity has been disrupted. In one embodiment, the the engineered cancer cell that produces less PD-L1 bearing exosomes comprises a cancer cell wherein nSMase2 expression or activity has been disrupted.

It will be understood that in some implementations, the engineered cancer cells of the invention may be modified to have both a reduced suppressive capacity and reduced EV production. For example, cells deficient in PD-L1 and Rab27a and/or nSMase2.

The aforementioned embodiments are directed to cancer cells engineered to have EVs with reduced suppressive capacity or having reduced EV production. “Engineering,” as used herein, encompasses any genetic modification of the cancer cell to modulate the expression or activity of one or more target genes. Engineering may further encompass the transformation of cancer cells to express one or more additional genes.

In certain embodiments, engineering comprises disrupting the expression of a target gene. Disruption of a selected target gene may be achieved various means known in the art. For example, any method known in the art for gene deletion, knockout, knockdown, or other means of reducing or ablating the expression of a target gene may be employed. Exemplary methods for reducing the expression of the target gene include the use of insertion mutagenesis, short interfering RNA, hairpin RNA, zinc finger nuclease, a transcription activator-like effector nuclease, or the use of CRISPR systems.

In other embodiments, engineering comprises modifications that interfere with the abundance or activity of a target protein. For example, the abundance of a target protein may be reduced by the co-expression of constructs that selectively target the protein for degradation, for example, a PROTAC or like construct. Another modification that affects target protein activity is the co-expression of a dominant negative mutant or other modulator of protein activity.

The engineered cancer cells of the invention may comprise additional modifications. In one embodiment, the additional modifications comprise the expression of factors which enhance immunogenic response against the cells, for example, the expression of cytokines, including GM-CSF, IL-18, IL-6, hyper IL-6, IL-11, hyper IL-11, IL15, and IL15α or antigens, such as cyclic di-guanylate, alphafetoprotien, carcinoembryogenic protein, CA-125, MUC1, epithelial tumor antigen, tyrosinase, and melanoma associated antigen.

In one implementation, the engineered cancer cells of the invention are modified in order to impair or limit the growth of the cells in order to prevent escape of administered cells, for example, by irradiation. In one embodiment, the engineered cells are modified to enable their inhibition in the event of escape, for example by conferring sensitivity to drugs, transformation with inducible suicide genes, or modifications to impair metastatic capacity.

As demonstrated herein, administration of the engineered cancer cells of the invention will invoke an immune response to like cancer cells. Thus, the engineered cells can be used to promote a subject's immune system to overcome systemic immunosuppression by resident tumors. The methods of the invention encompass any administration of a therapeutically effective amount of engineered cancer cells of the invention having impaired suppressive EVs to a subject in need of treatment, e.g., a subject having cancer. Therapeutically effective, as used in this context, means having any measurable enhancement of the immune system against resident cancer cells.

The vaccination procedure may be carried out by methods known in the art for the production and administration of cell-based vaccines. Exemplary methods for the preparation and administration of cellular vaccines are described in United States Patent Application Publication Number 20180008686, entitled “Autologous Tumor Vaccines and Methods,” by Hanna; Liu et al., 2018, Abscopal effect of radiotherapy combined with immune checkpoint inhibitors. J Hematol Oncol 11, 104; Gao et al., 2008, Secretable chaperone Grp170 enhances therapeutic activity of a novel tumor suppressor, mda-7/IL-24. Cancer Res 68, 3890-3898; Hurwitz et al., 2000, Combination immunotherapy of primary prostate cancer in a transgenic mouse model using CTLA-4 blockade. Cancer Res 60, 2444-2448; Morris, et al., 2014,. Vaccination with tumor cells expressing IL-15 and IL-15Ralpha inhibits murine breast and prostate cancer. Gene Ther 21, 393-401, Overwijk et al., 2003, Tumor regression and autoimmunity after reversal of a functionally tolerant state of self-reactive CD8+ T cells. J Exp Med 198, 569-580; and van Elsas et al., 1999, Combination immunotherapy of B16 melanoma using anti-cytotoxic T lymphocyte-associated antigen 4 (CTLA-4) and granulocyte/macrophage colony-stimulating factor (GM-CSF)-producing vaccines induces rejection of subcutaneous and metastatic tumors accompanied by autoimmune depigmentation. J Exp Med 190, 355-366.

The general method of production and administration encompasses formulating suppressive EV-deficient cells in a delivery composition and administering the delivery formulation to one or more sites. Delivery compositions may be administered by methods known in the art, for example, by injection, including intracorporeal injection, subcutaneous injection, intramuscular injection, or within the circulatory system. The administered engineered cells will encounter resident immune cells and provoke an immune response, wherein such immune response will attack resident cancer cells

Vaccine or vaccine-like compositions may encompass engineered cells in combination with adjuvants, carriers, excipients, and other elements utilized in whole cells vaccine formulations, as known in the art.

In some embodiments, the cells may be administered in or on physical elements which enhance their growth, enhance their immunogenic effects, and/or which limit their localized growth or mobility, for example, such as caging structures, scaffolds, nanoparticles, hydrogels, or other materials known in the art.

Generally, for enhanced immunogenic response, it is preferable that the administered cells be viable and grow or persist in the subject in order to provoke an immune response against resident tumors. In alternative embodiments, the administered cells will have impaired viability to prevent escape, for example, wherein the cells are irradiated or otherwise treated (e.g. by DNA crosslinking agents such as Mitomycin C, etc.) to reduce viability.

Vaccine compositions will be administered in a therapeutically effective amount. A therapeutically effective amount in this context means any amount that provokes an immune response, e.g. an immune response against the administered cells or against like cells resident or source tumors. As demonstrated herein, in some cases the balance of engineered to resident cancer cells is determinative of the efficacy of the method. Accordingly, in some embodiments, the engineered cells will be administered in sufficient amounts to overcome the suppressive effects of circulating PD-L1 exosomes or other suppressive EVs produced by the source tumor or other cancerous tissue. Such amount may be assessed by cell number, tumor size, or other measures of tumor growth. For example, the number of administered cells may be greater than the number of residual cancer cells in the subject, or the mass or volume of administered cells and may be greater than the mass/volume of resident or residual cancer cells. In some cases, the balance of immunogenic engineered cells to resident or residual cancer cells is determined by the size of the resulting tumor mass (or aggregate of two or more masses) created by the administered engineered cells, i.e. the progeny of the administered cells.

For example, in some embodiments, the number, mass, or volume of administered cells and/or their progeny may be at least 20%, at least 30%, at least 40%, at least 50%, at least 60%, at least 70%, at least 80%, at least 90%, at least 100%, at least 110%, at least 120%, at least 200%, at least 300% or at least 500% of the number, mass, or volume of resident cancer cells/tumors in the subject. Exemplary minimum dosages may be in the range of 1-100 million, or more, cells, for example, at least 10 million, at least 20 million, at least 50 million, at least 100 million, at least 500 million, at least one billion, at least five billion, or at least 10 billion cells. Exemplary maximum dosages may be in the range of 10 million to over 100 billion cells, for example, up to 10 million, up to 20 million, up to 50 million, up to 100 million, up to 200 million, up to 300 million, up to 400 million, up to 500 million, up to 1 billion cells, up to 5 billion cells, up to 10 billion cells, up to 50 billion cells, or up to 100 billion cells. Dosages may be administered once, or multiple dosages may be administered over a period of days, weeks, or months.

In one embodiment, in order to enhance the balance of engineered and/or exosome deficient cells to resident cancer cells in the subject, the vaccination treatment is administered in combination with treatments that impair, destroy, or reduce the numbers of resident cancer cells. For example, resident tumors may be treated with irradiation, surgery, or other treatments. For example, the therapeutic methods of the invention may be applied in combination with immunotherapy treatments, for example, CAR-T cell treatments, dendritic cell therapies, immunocheckpoint therapies, and other immunotherapies known in the art.

The vaccination methods of the invention may be applied in various contexts. In one embodiment, the vaccination method is applied in the treatment of resident cancer in the subject, for example, against one or more tumors present in the subject. In one embodiment, the vaccination method is applied as a preventative to inhibit the recurrence of a tumor by residual cancer cells or the progression of precancerous cells to cancer in the subject. For example, in a subject having a tumor, the tumor may be resected and the vaccine composition of the invention developed from the resected cancer cells, followed by administration of the vaccine composition to prime the subject's immune system against any residual tumor cells or the recurrence of the cancer.

Prognostic Methods. The aforementioned therapeutic methods provide novel means of treating cancer in subjects suffering therefrom. The scope of the invention further encompasses novel methods of assessing a subject's likelihood of responding to such treatment methods. The inventors of the present disclosure have determined that certain tumors are more responsive to the therapeutic methods of the invention, specifically, tumors having relatively fewer infiltrating immune cells. Currently, in the field of immunotherapy, the concept of immunologically “hot” and “cold” tumors has been established. Immunologically hot tumors are cancers wherein overall immune infiltrate is high and/or infiltrating immune cells are abundant, for example tumor infiltrating lymphocytes, whereas cold tumors have fewer infiltrating immune cells. Infiltrating immune cells include CD8⁺ T cells, CD3⁺ T cells FOXP3⁺ regulatory T cells (Tregs). In contrast, cold tumors contain fewer infiltrating immune cells, indicating a high level of immune suppression. Accordingly, such cold tumors are likely to respond to the treatments of the invention, which are directed to ameliorating or overcoming systemic suppression.

In a related implementation, the abundance of circulating suppressive EVs can be utilized as a measure of systemic suppression. According to the theory of the invention, responsiveness to immunotherapies is related to the degree of systemic suppression mediated by suppressive EVs in the subject. Accordingly, the abundance of circulating EVs serves as a measure of systemic suppression and may act as an indicator of which subjects are in need of relief from suppressive EV systemic effects.

Accordingly, in one implementation, the scope of the invention encompasses a method of treating cancer in a subject comprising the steps of:
assessing the degree of systemic suppression in the subject by a selected measure of systemic suppression; and
selecting a treatment regimen for the subject based on the assessed degree of systemic suppression, wherein subjects having substantial systemic suppression are administered a treatment to relieve such systemic suppression.

In one embodiment, the measure of systemic suppression is a determination if the subject has one or more cold tumors. In one embodiment, the measure of systemic suppression is a determination if the subject has substantial abundance of circulating suppressive EVs. In one embodiment, the measure of suppressive EVs is the abundance of circulating PD-L1-bearing exosomes.

In one embodiment, the treatment selected to suppress systemic suppression is the administration of a suppressive EV inhibitor. In one embodiment, the suppressive EV inhibitor is an inhibitor of PD-L1 bearing exosomes. In one embodiment, the inhibitor of PD-L1 bearing exosomes is an agent that reduces the expression or activity of Rab27a and/or snMase2. In one embodiment, the suppressive EV inhibitor is co-administered with an immunotherapy treatment. In one embodiment, the selected treatment to inhibit systemic suppression is the administration of engineered cancer cells deficient in suppressive EVs. In one embodiment, the engineered cancer cells are cancer cells having a reduced capacity to produce PD-L1 bearing exosomes.

Determination of “cold” status may be achieved by means known in the art for quantification of immune cells in a tumor sample. The tumor sample may be obtained by surgical excision or biopsy methods, as known in the art. The quantification of immune cells in the tumor sample may be achieved by methods known in the art, for example, immunohistochemical labeling, gene expression data (quantitative RT-PCR, gene expression arrays, RNA sequencing (both bulk and single cell)), or dissociation and cell sorting methodologies. Cold status may be determined according to threshold cutoff values relevant to the selected tumor and immune cell type, established in pools of like subjects with like cancers. In one implementation, the internationally established Immunoscore is utilized as a measure of immunologic status, as described in Pages et al, 2018, International validation of the consensus Immunoscore for the classification of colon cancer: a prognostic and accuracy study, The Lancet 391:2128-213.

Suppressive EV abundance may be measured by means known in the art. Circulating EVs such as exosomes may be assessed in a biological sample, for example, blood, serum, plasma, urine or lymph. The abundance of EVs in the sample may be assessed by methods such as size exclusion chromatography, ultracentrifugation, or precipitation reagents combined with detection of suppressive species (e.g. PD-L1), for example by fluorescent antibodies against suppressive molecules, for example high throughput quantitative microscopy techniques for detection of fluorescently labeled antibodies, ELISA, etc. The suppressive capacity and/or abundance of suppressive molecules, e.g. PD-L1, may be assessed by labeled antibodies, functional assays, or other means known in the art. For example, a Raji-Jurkat T-cell activation assay, as known in the art and as described in the Examples herein. Subjects are determined to have a high level of systemic suppression if the quantity of suppressive EVs, e.g., PD-L1 bearing exosomes, exceeds a selected threshold cutoff value established for such cancer and subject types.

Suppressive EV Quantification. The findings disclosed herein and elsewhere demonstrate that suppressive EVs, such as PD-L1 bearing exosomes, are utilized by cancer cells to evade the immune system. Accordingly, there is a need for methods of tracking and quantifying suppressive EVs. Advantageously, the inventors of the present disclosure have developed tools and methods for the visualization and quantification of suppressive EVs.

The EV quantification tools of the invention comprise fusion proteins. The fusion protein will comprise an EV-associated molecule fused to a reporter moiety. The fusion protein may be expressed in a cancer cell, wherein the reporter-labeled product will be loaded into and secreted within EVs, either presented on the membrane of the EV and/or within the cargo of the EV.

In one embodiment, the fusion protein may comprise any protein found in exosomes. For example, the general exosome marker may comprise CD63, CD81, CD9, TSG101, HRS, ALIX, or other species common to exosomes released by cancer cells. In another implementation, the labeled species comprises an immunosuppressive protein expressed in suppressive exosomes, for example, PD-L1, PD-1, CTLA-4 PD-1, PDL2, LAG3, TIM3, CD47, CD155, CD80, Gal-9, and MHC-Class I/II antigens, or other immunosuppressive species.

In one embodiment, the fusion protein is used for the visualization of suppressive microvesicles and the EV-associated species of the fusion protein may comprise any protein found in microvesicles.

The reporter moiety will comprise any protein reporter species that can be produced in a fusion product with the EV-associated protein, for example being joined at the c- or n-terminus of such protein. The reporter may comprise a fluorescent protein such as GFP, YFP or other fluorescent protein known in the art. The reporter may comprise an enzymatic reporter, such as a bioluminescence reporter, such as luciferase, nanoluciferase enzyme, or other enzymatic reporter known in the art.

Expression of the fusion protein may be engineered into cancer cell lines by means of corresponding nucleic acid constructs coding therefor, by methods known in the art. The fusion proteins may be expressed under the control of appropriate promoters, such as constitutive reporters. The fusion product is subsequently expressed in the cancer cell, packaged in EVs, and released to the extracellular environment. The transformed cancer cells may be cultured in vitro, or may be transplanted into living animals, for example, such as a tumor allografts or xenografts in test animal. In one embodiment, the expression of the fusion protein in specific cell types that give rise to tumors is engineered into a whole organism, wherein, if cancer arises, exosome reporter fusion proteins produced by the cancer will contain the expressed fusion protein.

Any type of cancer cell may be transformed to express the fusion proteins of the invention, including human and non-human cancer cell lines. In one embodiment, the fusion protein is expressed in a prostate cancer cell line. In one embodiment, the prostate cancer cell line comprises a PC3, SK-MEL 28, DU145 or TRAMP cell line. Additional cell lines that may be used include NCI60 cell lines, as known in the art.

In various embodiments, the scope of the invention encompasses nucleic acids coding for the fusion proteins of the invention, the fusion proteins themselves, cells expressing the fusion proteins, and EVs containing the fusion protein.

The fusion proteins of the invention enable the tracking and/or quantification of suppressive EVs by visualization or quantification of the fusion protein. Such visualization or quantification will be performed in a sample. In one embodiment, the fusion protein is expressed in cultured cells and the sample comprises growth media or cell culture exudate. In one embodiment, the fusion proteins of the invention are expressed in allografted or xenografted cancer cells, or in animals expressing the fusion protein in cancer cells. In such embodiments, the sample may comprise blood, plasma, lymph, or immune compartment tissues such as the lymph nodes, or tumor biopsy material. In one embodiment, the sample is a tissue section.

The presence and/or abundance of suppressive EVs can be observed by the use of an appropriate assay for visualization and/or quantification of the reporter, such as by fluorescence microscopy or bioluminescence assay, as the case may be.

In one implementation, the fusion protein biomarker of suppressive exosomes is assessed in a screening protocol for the identification of or optimization of agents that inhibit the formation, release, or activity of suppressive exosomes. In another implementation, in whole animals, the fusion protein is utilized to track the trafficking and distribution of suppressive exosomes, for example, in the lymph system.

Suppressive EVs as Immunosuppressive Agents. Based on the observed immunosuppressive properties of circulating PD-L1 exosomes, the inventors of the present disclosure have developed compositions and methods for systemic suppression of immune processes in a subject. The suppressive effects of circulating suppressive EVs produced by cancer cells disclosed herein can be harnessed for the treatment of conditions associated with immune activity. Exogenously produced suppressive EVs, referred to herein as therapeutic suppressive EVs, can be administered to a subject to promote systemic downregulation of the immune system or treating an immune-related condition. As used herein, an immune-related condition comprises an inflammatory condition or autoimmune condition, or the need for immunosuppression, for example, the need for immunosuppression associated with the receipt of a transplant.

In one embodiment, the inflammatory or autoimmune condition is selected from the group consisting of arthritis (e.g., rheumatoid arthritis), multiple sclerosis, inflammatory bowel disease, Crohn disease, lupus, autoimmune uveitis, type I diabetes, bronchial asthma, lupus, retinitis, pancreatitis, cardiomyopathy, pericarditis, colitis, glomerulonephritis, lung inflammation, esophagitis, gastritis, duodenitis, ileitis, meningitis, encephalitis, encephalomyelitis, transverse myelitis, cystitis, urethritis, mucositis, lymphadenitis, dermatitis, hepatitis, osteomyelitis, psoriasis, scleroderma, dermatomyositis, epidermolysis bullosa, and bullous pemphigoid.

Regarding the need for immunosuppression, the need may be applicable to a subject prior to or following receipt of a transplant, wherein the transplant may comprise any graft, for example, a graft selected from the group consisting of of an organ, tissue, cells, kidney, heart, lung, liver, skin, cornea, intestine, pancreas, limb, digit, bone, ligament, cartilage, and tendon.

In one aspect, the scope of the invention encompasses a therapeutic immunosuppressive EV for use in the treatment of an immune-related condition. The therapeutic immunosuppressive EV may comprise an EV bearing one or more immunosuppressive molecules. The immunosuppressive EV is administered in a therapeutically effective amount to a subject in need of treatment of an immune-related condition. In one embodiment, the scope of the invention encompasses the use of a therapeutic immunosuppressive EV in the manufacture of a medicament for the treatment of an immune-related condition

The therapeutic suppressive EVs of the invention will comprise one or more immunosuppressive molecules. The immunosuppressive molecules may be present in the EV membrane and/or in the EV cargo. In a first embodiment, the one or more immunosuppressive molecule comprises one or more immune checkpoint molecules. In one embodiment, the one or more immunosuppressive molecule comprises PD-L1, wherein such will be referred to as a therapeutic PD-L1 exosome. In other embodiments, the one or more immunosuppressive molecules may comprise, for example, PD-1, PD-2, adenosine A2A receptor, B7-H3 (CD276), B7-H4 (VTCN1), BTLA, CTLA-4, Indoleamine 2,3-dioxygenase, Killer-cell Immunoglobulin-like Receptor, Lymphocyte Activation Gene-3,NOX-2, PD-1, TIM-3, or V-domain Ig suppressor of T cell activation.

In other embodiments, the one or more immunosuppressive molecule may comprise an immunosuppressive peptide, for example, Antamides, Collutellin A, Cyclosporine A, Didemnin A/B, FK506 (tracrolimus), Ascomycin (pimecrolimus, SDZ ASM 981), Homophymines, Geodiamolides H, Hymenistatin, Charybdotoxin I, Curcacycline B, Cyclolinopeptide A/B Iberiotoxin, Kalata B1, Magatoxin, and Kaliotoxin.

In one embodiment, the immunosuppressive molecule may comprise an antibody or fragment thereof which inactivates a receptor implicated in immune response. In one embodiment, the immunosuppressive molecule may comprise a small molecule immunosuppressive drug or steroid.

In one embodiment, the EV is engineered to display binding moieties on its surface, enabling post-secretion functionalization with immunosuppressive drugs or other compositions. Exemplary methods of functionalizing EVs are described in Smyth et al., 2014, Surface Functionalization of Exosomes Using Click Chemistry, Bioconjug Chem 25:1777-1784.

The one or more immunosuppressive molecules, e.g. PD-L1, may be present in any biologically effective amount, for example, tens, hundreds, thousands (e.g. at least or up to 10,000, at least or up to 50,000, at least or up to 100,000 immunosuppressive molecules per vesicle), millions (e.g. at least or up to 10 million, at least or up to 20 million, at least of up to 50 million, at least or up to 75 million, at least or up to 100 million, or at least or up to 500 million immunosuppressive molecules per vesicle), or billions (e.g. at least or up to 1 billion, at least or up to 5 billion, at least or up to 10 billion immunosuppressive molecules per vesicle).

In the case of immunosuppressive molecules comprising proteins (e.g., PD-L1), the protein may comprise a wild type protein, or may comprise a mutant or engineered protein, or a biologically active protein fragment or fusion protein, e.g. comprising an active extracellular domain.

The suppressive EVs of the invention encompass any extracellular vesicle capable of displaying and/or carrying suppressive molecules and promoting the downregulation or inhibition of one or more immune responses. In a first embodiment, the EV will comprise an exosome, being an intracellularly produced EV. In a second embodiment, the EV will comprise a microvesicle produced and budded from the plasma membrane. In an alternative embodiment, the EV is a synthetic vesicle.

The therapeutic immunosuppressive EV may be of any size, for example, in the range of 10 nm-200 μm. In one embodiment, the immunosuppressive exosomes are derived from the cells of the subject to which the exosomes will be administered. In another embodiment, the immunosuppressive exosomes are generic or universal exosomes derived from cells of another subject, wherein the generic exosomes are non-immunogenic and compatible with the subject to which to which they will be administered.

The production of EVs, e.g., exosomes, may be accomplished by the use of cultured cells, for example, autologous cells derived from a subject to which the therapeutic suppressive EVs will be administered. The cultured cells may be genetically transformed, by mean known in the art, to express one or more immunosuppressive molecules which are packaged into exosomes or other EVs produced by the cultured cells. For example, cells engineered to express PD-L1 on exosomes may be utilized. Suppressive EVs produced by the cultured cells may be harvested by means known in the art, for example by collection of cell culture liquid medium followed by filtration, centrifugation, or other EV isolation steps known in the art.

The production of customized EVs is known in the art. Exemplary sources of therapeutic suppressive EVs include, for example, cultured cells, for example, cultured human cells, for example, cultured human cancer cells. Additional exemplary sources of therapeutic suppressive EVs may include compositions of matter as described in Li et al., Exosomal cargo-loading and synthetic exosome-mimics as potential therapeutic tools, Acta Pharmacologica Sinica volume 39, pages 542-551 (2018); Kooijmans et al., Exosome mimetics: a novel class of drug delivery systems, Int J Nanomedicine, 2012; 7: 1525-1541; Conlan et al., Exosomes as Reconfigurable Therapeutic Systems, Trends Mol Med 23:636-650 (2017); U.S. Pat. No. 9,480,714, entitled “Methods and systems for processing exosomes” by Wu et al.; U.S. Pat. No. 7,704,964, entitled “Methods and compounds for the targeting of protein to exosomes,” by DelCayre and LePeq; and PCT International Patent Application Number WO2016172598, entitled “Exosomes and uses thereof, by TerOvanesyan et al.

The general therapeutic method of the invention is as follows:
a method of suppression of the immune system of a subject in need of immunosuppressive treatment, comprising
the administration of a therapeutically effective amount of a suppressive EV bearing one or more immunosuppressive molecules to the subject.

The subject may be suffering from an immune-related condition, for example, an autoimmune disorder, for example, an autoimmune disorder such as arthritis, inflammatory bowel disease, Crohn's disease, or ulcerative colitis, lupus, progressive systemic scleroderma, mixed connective tissue disease and antiphospholipid syndrome, multiple sclerosis, and diabetes. The subject may be a subject about to receive or that has received a graft or transplant, for example being administered to prevent rejection, for example as pre- or post-transplant immunosuppression, maintenance suppression, or to treat acute rejection processes. Exemplary transplants include kidney, liver, lung, bone marrow, skin grafts, blood vessels, and other grafts known in the art. In one embodiment the subject is a subject suffering from a chronic inflammatory condition.

The therapeutic immunosuppressive EVs may be administered at any dosage which produces a measureable suppression of immune function, as measured by methods known in the art. Suppression of immune function, includes, for example, suppression of one or more selected immune responses, inhibition of one or more immune cell activities, a reduction or ablation of one or more inflammatory processes, modulation of immune cell numbers (e.g. absolute or relative numbers), inhibiting the priming of T-cells, or promotion of any-PD-L1 mediated process.

Therapeutic suppressive exosomes may be administered at any dosages, for example, 1 ng-200 mg exosome (as measure by protein content or total exosome mass), for example, at least 10 ng, at least 100 ng, at least 1 μg, at least 10 μg, at least 100 μg, at least 1 mg, or at least 10 mg. For example, in one implementation, a dosage of 2-20 mg exosomes may be administered for an average 70 kg human.

Alternatively, dosages may be expressed as exosomes produced by source cells per day, for example as produced by 1 million to 50 billion cells per day, for example, exosomes produced by about 10 million, 20 million, 50 million, 100 million, 500 million, 1 billion, 10 billion or 50 billion cells per day, for example, the exosome output of 5-50 billion cells per day, per administration.

Administration may be intravenous, intramuscular, subcutaneous, or by any other means which exposes the administered exosomes to immune cells of the subject. For systemic suppression, administration to the circulatory or lymph system may be employed. For localized suppression, administration to the target area may be employed.

In exemplary embodiments: The scope of the invention encompasses exogenously produced suppressive EVs for use in a method of treating an immune-related condition. In one embodiment, the exogenously produced suppressive EV is an exosome comprising PD-L1. In one embodiment, the invention encompasses suppressive EVs for use in a method of treating an autoimmune disorder. In one embodiment, the invention encompasses suppressive EVs for use in a method of preventing or treating transplant rejection. The scope of the invention further encompasses methods of making an immunosuppressive medicament by the use of suppressive exosomes, for example, PD-L1-bearing exosomes.

in another aspect, the scope of the invention encompasses a pharmaceutical composition for use in the treatment of an immune-related condition, the pharmaceutical composition comprising therapeutic suppressive EVs (e.g., exosomes) comprising one or more suppressive molecules, for example PD-L1. The pharmaceutical composition may further comprise pharmaceutically acceptable carriers, such as buffers, preservatives, suspending, stabilizing and/or dispersing agents, and other pharmaceutically acceptable carriers that facilitate the preservation and delivery of the therapeutic exosomes. In one embodiment, the pharmaceutical composition is a liquid. In one embodiment, the pharmaceutical composition comprises a lyophilized powder or dry formulation.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

### EXAMPLES

### Example 1

### Inhibition of Suppressive Exosomes

Disclosed herein is a novel pathway that regulates the presentation of immune checkpoints. In particular, the immune checkpoint ligand PD-L1 can be presented on exosomes along with its known presentation on the plasma membrane of cells, for example, as disclosed herein, exosomes are a major source of PD-L1 presentation in the prostate cancer cell line PC3. Exosomes are small vesicles released by a cell, which, unlike cells, can travel great distances throughout the body. Therefore, presentation of PD-L1 on exosomes can result in immune suppression distally as well as locally in the tumor bed, greatly increasing its potential potency. By genetically targeting enzymes that regulate exosome biogenesis, exosomal presentation of PD-L1 was largely blocked in vitro and in vivo. In an in vivo model of prostate cancer, blocking exosomal PD-L1 suppressed tumor progression and increased survival. Importantly, this model has previously been shown to be resistant to antibody blockade of PD-L1, proving that current therapeutic strategies fail to fully inhibit this critical immune checkpoint regulator.

Given that suppressing the exosomal presentation of PD-L1 would be orthogonal to current treatments, its modulation has the potential to function alone as well as act in concert with current immune checkpoint therapies.

Evidence presented herein shows that deletion of either Rab27a or nSMase2 blocks secretion of PD-L1 from tumor cells and suppresses tumor progression in vivo. Also disclosed herein are high-throughput cell assays for both exosome production and PD-L1 secretion.

The PD-1-PD-L1 pathway is generally thought to function within the tumor bed(s) to suppress immune attack of malignant cells. In particular, malignant cells upregulate PD-L1 on their surface, which then interacts with the PD-1 receptor on cytolytic T cells. This interaction blocks activation and proliferation of the T cells, thereby suppressing T cell-directed killing of the tumor cells. PD-L1 has also been found on antigen presenting cells, similarly suppressing T cell activation. While the focus in the field has previously been on cell surface presentation of PD-L1, herein it is disclosed that a large portion of PD-L1 is secreted from cells in small vesicles called exosomes. The PD-L1 on the surface of the exosomes is able to suppress T cells similar to cell surface PD-L1. However, exosomes can travel far from the cell, inducing broader suppression both in the tumor itself and also throughout the body, potentially inducing a systemic immunodeficient state. As such, exosome presentation of PD-L1 has the potential to have a much greater impact on the body's response to cancer than the PD-L1 found on the surface of tumor cells.

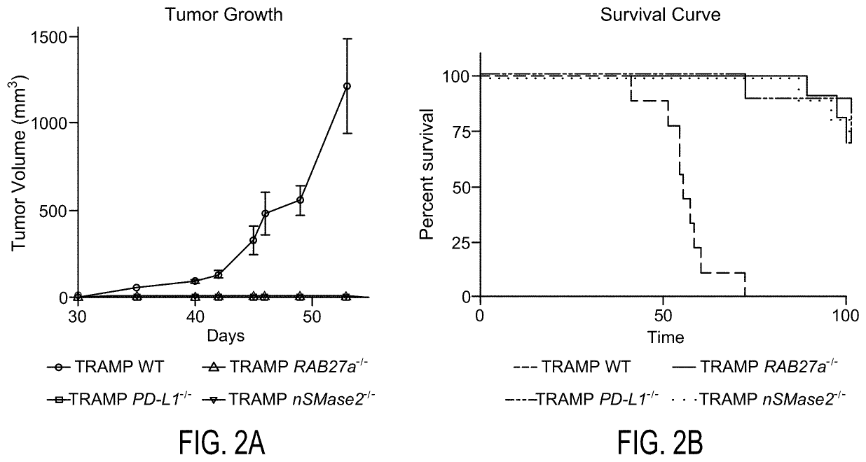

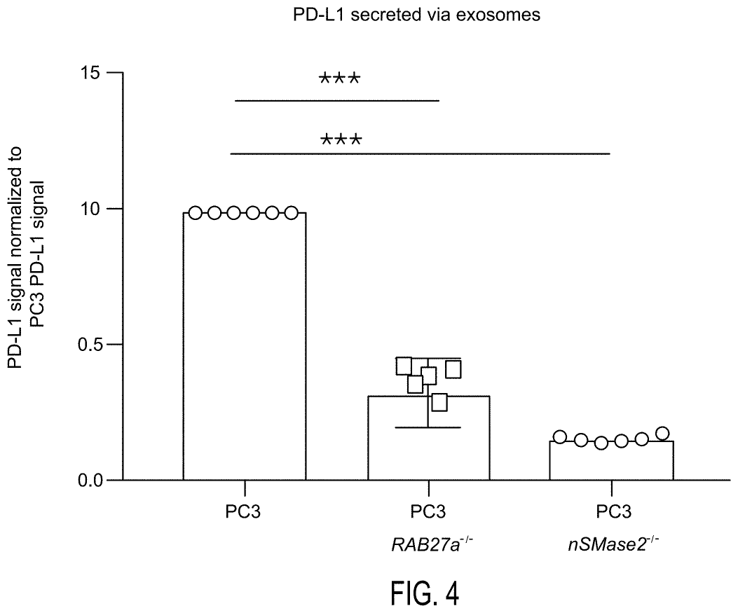

Exosomes arise from a carefully choreographed process within cells. The plasma membrane of cells goes through a process of internal pinching called endocytosis. The resulting endosomes can then either be recycled to the membrane or mature into late endosomes. During the maturation process to late endosomes, the surrounding (or limiting) membrane pinches internally to form small vesicles called intraluminal vesicles. The resulting late endosomes carry multiple intraluminal vesicles and thus are called multi-vesicular bodies (MVBs). The MVBs can either fuse with lysosomes resulting in the degradation of their contents or with the plasma membranes resulting in the release of the internal vesicles, which are then termed exosomes. As demonstrated herein, tumor cells can use this biogenesis process to transport plasma membrane bound PD-L1 to the surface of exosomes. A number of enzymes have been identified in the exosome biogenesis pathway, including nSMase2 and Rab27a. By deleting the genes encoding these enzymes, secretion of exosomal PD-L1 is blocked (FIG. 4). Furthermore, using a syngeneic mouse model of prostate cancer, it was observed that deleting either the gene encoding PD-L1, Rab27a, nSMase2 dramatically suppresses tumor growth and extends long term survival (FIGS. 2A and 2B). Remarkably, this tumor model has previously been shown to be resistant to existing anti-PD-L1 antibody blockade therapy. These data demonstrate that exosomal PD-L1 is effective in suppressing immune destruction of tumor cells and is resistant to current anti-PD-L1 therapies. Thus, an anti-exosomal PD-L1 therapy will increase the efficiency of PD-L1 blockade and tumor response to treatment.

It is demonstrated herein that that the deletion of two known regulators of exosome biogenesis, nSMase2 and Rab27a blocks the secretion of PD-L1

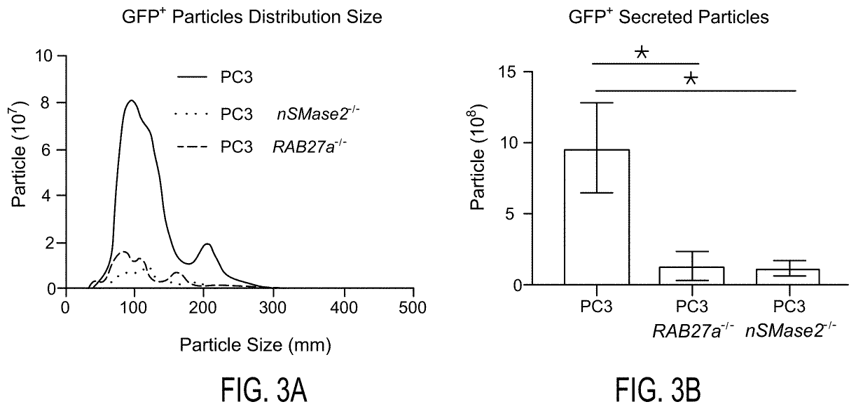

Exosome Tracking and Quantification To quantitatively evaluate exosome production, PC3 cell line was transduced with a CD63-GFP transgene. A nanoparticle tracker was used to simultaneously measure the size and fluorescence of vesicles released into the media of the cultured cells. This analysis confirmed a dramatic decrease in the number of GFP+ exosome-sized particles secreted from both the Rab27a and nSMase2 knockout cells, confirming the sensitivity and specificity of this approach to quantitatively measure inhibition of the two enzymes (FIGS. 3A and 3B).

As described above, by combining the CD63-GFP tool together with the Nanosight nanoparticle tracking system, it is possible to screen compounds for anti-exosomal activity. A similar construct was made, replacing the GFP with a gene encoding a nanoluciferase enzyme (Nluc). Constructs that have Nluc fused to either the N-terminal or C-terminal end of PD-L1 were also made. Packaging of the C-term PD-L1-Nluc fusion protein into exosomes from WT PC3 cells was confirmed and the constructs were introduced into the nSMase2KO and Rab27aKO cell lines to validate the system. Luciferase signal was observed from the secreted PD-L1-Nluc in a 96-well format, optimizing cell density and culture time for the greatest differential between the wt and knockout lines. All luciferase measurements of the media were normalized to luciferase in the cells to correct for any differences in cell number or transgene expression.

Suppression of PD-L1-Nluc alone, PD-L1-Nluc and CD63-Nluc, or CD63-Nluc alone may be used to assess anti-exosomal compounds. For example, PD-L1-Nluc alone inhibited the loading of PD-L1 into exosomes without altering other functions of exosomes. Hits may be prioritized based on the degree of suppression, minimal cell toxicity, common chemical structures with other hits, known activities, and predicted pharmacokinetic and pharmacodynamic properties.

### Example 2

### Suppression of PD-L1 Secreted in Exosomes Promotes Systemic Anti-Tumor Immunity and Memory

Summary: Antibody blockade of the immune checkpoint protein PD-L1 leads to durable remissions in a subset of cancer patients. PD-L1 is thought to act in the tumor bed by binding its receptor PD-1 on effector T-cells. Here is described an alternative role and susceptibility for PD-L1 secreted by tumor cells in the form of exosomes. Removal of exosomal PD-L1 inhibits tumor growth, even in models resistant to anti-PD-L1/PD-1 antibodies. Exosomal PD-L1 released from the tumor suppressed T-cell activation in the draining lymph node. Systemically introduced exosomal PD-L1 rescued growth of tumors unable to secrete their own. Exposure to exosomal PD-L1 deficient tumor cells suppressed growth of wild-type tumor cells injected at a distant site, simultaneously or many months later. Anti-PD-L1 antibodies worked additively, not redundantly, with exosomal PD-L1 blockade to suppress tumor growth. Together, these findings show that inhibition of exosomal PD-L1 can overcome systemic suppression mediated by suppressive EVs and resistance to current antibody approaches.

EVs are heterogeneous. A particular form of EVs are exosomes, which derive from the endocytic pathways. As endosomes mature, vesicles bud inward and are released in the lumen forming intravesicular bodies within the late endosomes. These late endosomes are also called multivesicular bodies (MVB). MVBs can either fuse with lysosomes for degradation and recycling of contents or fuse with the plasma membrane releasing the intravesicular bodies extracellularly, which are then called exosomes. Exosomes can be differentiated from other EVs based on their size, morphology, density, marker expression, and dependency for specific enzymes for their biogenesis. Key enzymes in their biogenesis include NSMASE2, which promotes budding of intravesicular vesicles, and RAB27A, which is involved in the fusion of the MVB to the plasma membrane. Genetic manipulation of these enzymes provides an opportunity to dissect the role of exosomes in vivo.

As above, it is shown that cancer cells can secrete a vast majority of their PD-L1 within exosomes rather than present PD-L1 on their cell surface. Using genetic knockouts for Rab27a and nSMase2 and exogenously introduced exosomal PD-L1, it was demonstrated that exosomal PD-L1 from tumor cells suppress T cell function in vitro and in vivo and promote tumor growth in an immune-dependent fashion. Exosomal PD-L1 appears to be resistant to anti-PD-L1/PD-1 as a prostate cancer syngeneic model that is unresponsive to such therapy, is dependent on both PD-L1 and exosomes for their growth. Remarkably, even the transient presence of cancer cells deficient in exosomal PD-L1 results in long-term, systemic immunity against the cancer. A role for exosomal PD-L1 is also seen in a syngeneic colorectal model. In this model, anti-PD-L1 acts additively, not redundantly, with the suppression of PD-L1 secretion. These findings have significant implications for immunotherapeutic approaches to cancer therapy.

Differential secretion of PD-L1 between cancer cell lines. It has been reported that surface PD-L1 levels are low in prostate cancer cell lines and primary prostate tumor tissue, potentially explaining the general lack of therapeutic response to anti-PD-L1 blockade. To determine whether a transcriptional, post-transcriptional, translational, or post-translational mechanism underlies the reduced levels, prostate cancer cell lines (P3, DU145, LNCaP) to a melanoma cell line (SK-MEL-28) were compared. Measurements of mRNA and protein levels showed a discordance between mRNA and protein levels across the different cancer cell lines. Reverse transcriptase-quantitative PCR (RT-qPCR) showed a 15-fold increase of Pd-l1 mRNA levels in PC3 and DU145 relative to SK-MEL-28; LNCaP showed a near absence of transcripts. In contrast to mRNA levels, western analysis showed similar cellular PD-L1 protein levels in PC3 and DU145 cells as SK-MEL-28; protein was undetectable in LNCaP cells. It was explored whether the discordance in mRNA and protein levels between PC3 and SK-MEL-28 could be explained by differences in protein translation. Translation rates were determined by polysome profiling, a method where transcripts bound by many ribosomes reflective of a high translation rate are separated from transcripts bound by one or a few ribosomes reflective of a low translation rate. The two populations were separated on a sucrose gradient and then the bound mRNA was measured by RT-qPCR. This analysis showed an equal distribution of the Pd-l1 RNA across the fractions in PC3 and SK-MEL-28. Thus, differences in translation rates cannot explain the discordance between mRNA and protein levels between the lines.

Next, potential differences in protein degradation were evaluated. The two main pathways for protein degradation are the lysosome and the proteasome. The small molecule Bafalomycin A1 (BafA1) inhibits lysosomal activity by blocking the V-ATPase hydrogen pump and thus acidification of the lysosome. An increase in the protein LC3B, a known target of the lysosome, confirmed effectiveness of the small molecule on PC3 and SK-MEL-28 cells. However, levels of PD-L1 in both lines were unaltered, implying little turnover of PD-L1 by the lysosome in these cells. The small molecule MG132 suppresses proteasome activity by blocking the 26s proteasome complex and consequentially proteolysis. The proteasome recognizes ubiquitin side chains on its targets. An increase in the presence of ubiquitinated proteins confirmed the effectiveness of MG132 on the two cells lines. Again though, PD-L1 protein levels were minimally affected and, if anything, the difference of the two lines was opposite from expected as PD-L1 levels were slightly up in SK-MEL-28, but not PC3 cells. These results show that differences in protein stability do not explain the discordance in mRNA and protein levels.

Next, the possibility that PD-L1 may be differentially secreted from cells in the form of membrane vesicles was considered. Extracellular vesicles can be enriched using sequential centrifugation transferring supernatant through increasing gravitational forces to remove cellular debris and apoptotic bodies, before finally pelleting at 100,000× gravitational force (100 k g). Western analysis showed two to three-fold more PD-L1 in the 100 k g fraction of PC3 cells relative to SK-MEL-28 cells. This difference could have been due to more PD-L1 being loaded per vesicle or release of more vesicles. A nanoparticle tracking instrument can track the size and number of vesicles by using light diffraction off particles moving under Brownian motion. Analysis of conditioned media from the two cell types showed equal total vesicle counts. Thus, even though PC3 and SK-MEL-28 cells had similar levels of cellular PD-L1 protein, PC3 cells packaged greater amounts of PD-L 1 into extracellular vesicles. This difference appears to underlie the discordance between mRNA and protein levels between the two cell lines.

In vivo, tumor cells can mediate adaptive resistance by up-regulating PD-L1 in response to interferon-gamma (IFNγ) released by the cytotoxic T lymphocytes within the tumor bed. Therefore, it was explored whether, in addition to up-regulating cellular PD-L1, IFNγ may increase the secretion of PD-L1. Treatment of cancer cells with IFNγ led to an increase in PD-L1 in both the cellular and 100 k g fractions. The increase was proportionally similar between the two fractions suggesting no direct impact of IFNγ on PD-L1 secretion. In addition, IFNγ did not increase the number of vesicles secreted. Thus, similar to the cell surface membrane PD-L1, extracellular vesicular PD-L1 increased in response to IFNγ.

PD-L1 is specifically secreted within exosomes. Extracellular vesicles come in multiple forms differing in size, density, protein markers, and biogenesis. Given that PD-L1 is endocytosed from the surface of cells, it was hypothesized that PD-L1 is being specifically secreted in the form of exosomes. Exosomes can be enriched relative to other vesicles based on their density by spinning the crude 100 k g pellet on a sucrose gradient. The exosomal marker CD63 traveled in the 20-40% sucrose fractions. PD-L1 and the additional exosomal marker HRS colocalized with CD63. These data support that PD-L1 is packaged in exosomes.

Next, to determine if PD-L1 is specifically found in exosomes, a genetic approach to remove exosomes was utilized. Rab27a and nSMase2 genes were knocked out in PC3 cells using CRISPR/Cas9-mediated mutagenesis. Deletion of these two genes did not effect the proliferation of PC3 cells. In order to follow exosomes, CD63-GFP was ectopically expressed in the three genetic backgrounds. Measurement of the conditioned media showed an almost complete absence of CD63-GFP+ particles arising from the two mutant lines vs. WT. To further evaluate the effect of the Rab27a and nSMase2 deletion on exosomes, electron microscopy was performed on sucrose gradient concentrated particles. The resulting images showed very few exosome-like particles in the Rab27a and nSMase2 null samples, with a greater loss in the Rab27a null cells. Consistent with these images, western analysis showed an absence of endogenous CD63 in the 100K g preps from Rab27a null cells and a small amount remaining in the nSMase2 null cells. In contrast to CD63 levels, nSMase2 showed a complete absence, while Rab27a null cells showed a dramatic reduction in PD-L1 in the 100K g fraction. This difference appears to be due to an additional role for NSMASE2 on Pd-l1 transcription. Together, these data show critical roles for both Rab27a and nSMase2 in exosome biogenesis and PD-L1 secretion.

Given that PD-L1 is a trans-membrane protein, exosomal PD-L1 must arise from the limiting membrane of the late endosome. The limiting membrane of the late endosome arises initially from the endocytosis of the plasma membrane. However, material provided directly from the ER and Golgi as well as cytoplasm is added as the resulting endosome matures. Flow cytometry and immunofluorescence showed the presence of PD-L1 on both the surface and within vesicle-like structures inside PC3 cells. Using standard cell fractionation techniques, PD-L1 and CD63 co-localized in the sucrose light, or endolysosomal fractions. To determine whether exosomal PD-L1 arises directly from the ER or early Golgi, the fact that PD-L1 is glycosylated was exploited. This glycosylation was easily removed with the amidase PNGaseF, consistent with it being a N-linked oligosaccharide chain. In contrast, the majority of cellular and all of the exosomal PD-L1 was resistant to EndoH cleavage, consistent with maturation of the oligosaccharide chain in the distal Golgi. Therefore, exosomal PD-L1 does not appear to come directly from the ER or early Golgi. To measure the plasma membrane as a source, a cell surface biotinylation assay was performed. Biotin-labeled PD-L1 was found in the cells and exosomes of treated cells, showing that exosomal PD-L1 originates from the surface of the PC3 cells.

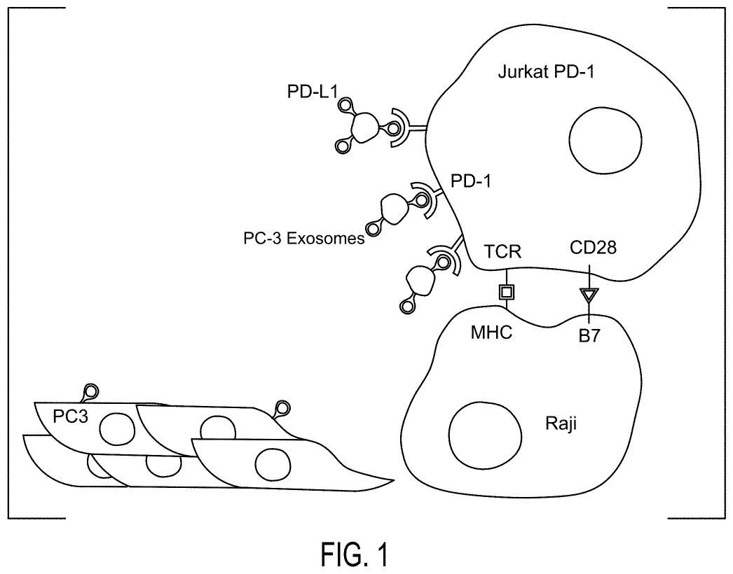

Exosomal PD-L1 suppresses T cell activation. Next, it was explored whether exosomal PD-L1 could function similarly to cell surface PD-L1 in the suppression of T cell activation. PD-L1 function can be measured in vitro in the setting of Raji B cell presentation of antigen to Jurkat T cells, as known in the art. Normally, this presentation would activate T cells, which can be measured by secretion of interleukin-2 (IL-2). However, if PD-L1 is exogenously expressed in the Raji B cells and PD-1 is exogenously expressed in the Jurkat T cells, T cell activation is suppressed (FIG. 1). In this setting, it was explored whether exosomal PD-L1 from PC3 cells could replace exogenous expression of PD-L1 on the Raji B cells. As expected, in the absence of exogenous expression of PD-L1 in Raji B cells, IL-2 secretion was high. However, the introduction of the PC3 100K g extracellular fraction re-repressed IL-2 secretion showing that PC3 vesicles can replace Raji cell PD-L1 expression. To determine whether exosomal PD-L1 was responsible for this effect, CRISPR/Cas9 editing was used to delete the Pd-l1 gene in the PC3 cells. Deletion of the Pd-l1 gene had no negative consequence on the proliferation of the PC3 cells, nor did it alter the number of vesicles released. However, introduction of the Pd-l1 null PC3 100K g extracellular fraction failed to repress IL2 secretion. These data show that exosomal PD-L1 does function to suppress T cell activation in vitro.

Exosomal PD-L1 promotes tumor progression. Given its ability to suppress T cell activation in vitro, it was explored whether exosomal PD-L1 can function in vivo to promote tumor progression. A syngeneic model of prostate cancer, the TRAMP model was employed. This preclinical model, like human prostate cancer, is known to be resistant to anti-PD-L1 anti-PD-1 blockade. CRISPR/Cas9-mediated deletion of Rab27a and Pd-l1 resulted in a loss of PD-L1 in the EV fraction. Loss of Rab27a did not influence cell surface PD-L1 levels, nor did loss of Pd-l1 influence exosome production. Like PC3 cells, deletion of Rab27a or Pd-l1 had no impact on the proliferation of the cells. The wt, Rab27a null, Pd-l1 null TRAMP cells were injected into the flanks of C57BL6/j syngeneic mice and were followed for over four months. All mice injected with the WT TRAMP cells had visible tumors by around 35 days and had to be euthanized between 40 and 71 days. In contrast, all mice injected with Rab27a null or Pd-l1 null TRAMP cells showed no tumor growth during the same time period (FIG. 2A). Similarly, the mice injected with Rab27a and Pd-l1 null TRAMP cells showed a dramatically extended lifespan relative to their wildtype counterparts (FIG. 2B). Indeed, a majority of the mice remained alive after 90 days, at which point they were used for memory experiments described below.

To further confirm that the loss of Rab27a was blocking tumor growth through its role in exosome biogenesis, the experiments were repeated using nSMase2 null cells. nSMase2 was deleted again using CRISPR/Cas9-mediated mutagenesis. Similar to PC3, nSMase2 null TRAMP cells showed a decrease in both cellular and extracellular levels of PD-L1. These cells were injected in syngeneic mice and compared to their WT counterparts. Similar to Rab27a null TRAMP cells, the nSMase2 null TRAMP cells failed to form palpable tumors during the time period when mice receiving WT cells had to euthanized (FIG. 4A). Again, a majority of the mice (8 out of 10) were still alive after 90 days. Together, these data show that the removal of factors important in exosome biogenesis or PD-L1 result in very similar tumor growth suppression phenotype.

It remained plausible that the exosome biogenesis factors were acting cell autonomously to suppress tumor growth, rather than non-autonomously via exosomal PD-L1 to suppress tumor growth via the anti-tumor immune response. To differentiate these alternative possibilities, it was explored whether the effect of Rab27a and/or nSMase2 loss was dependent on an active immune system. WT, Rab27a, nSMase2, and Pd-l1 null TRAMP cells were injected into NOD-scid NSG immunodeficient mice. In striking contrast to results seen in immunocompetent mice, the four lines led to end-stage tumors at an identical rate in the immunodeficient background. Therefore, RAB27A and NSMASE2, like PD-L1 are promoting tumor growth through the suppression of the immune system.

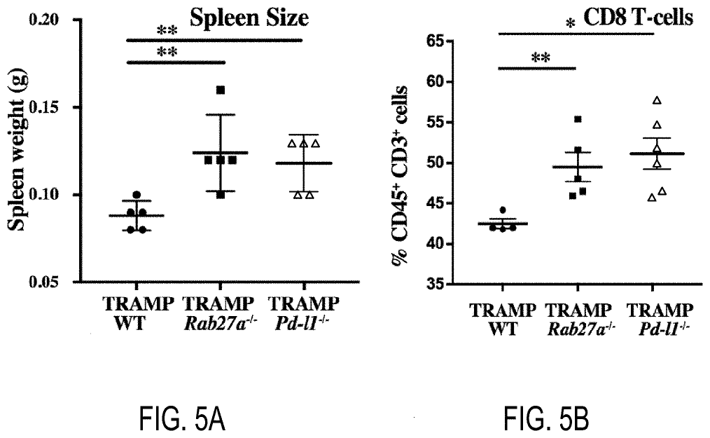

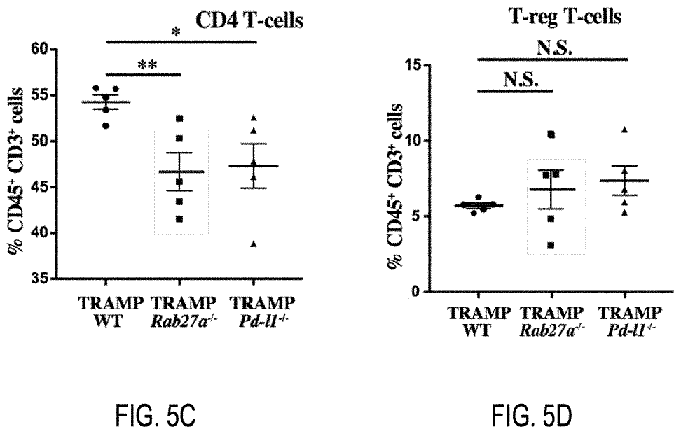

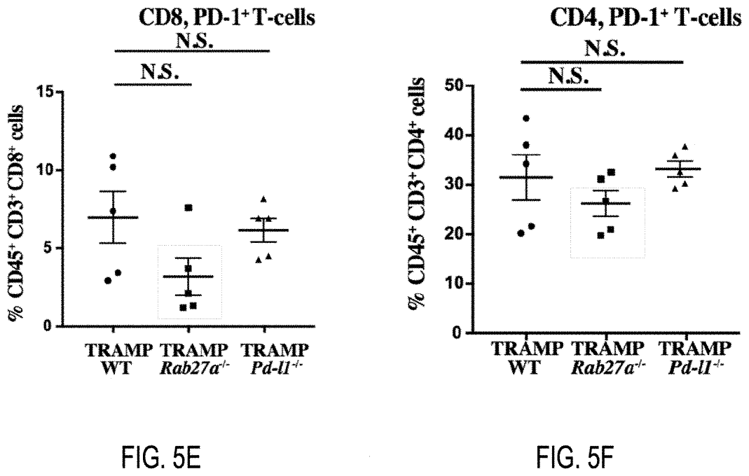

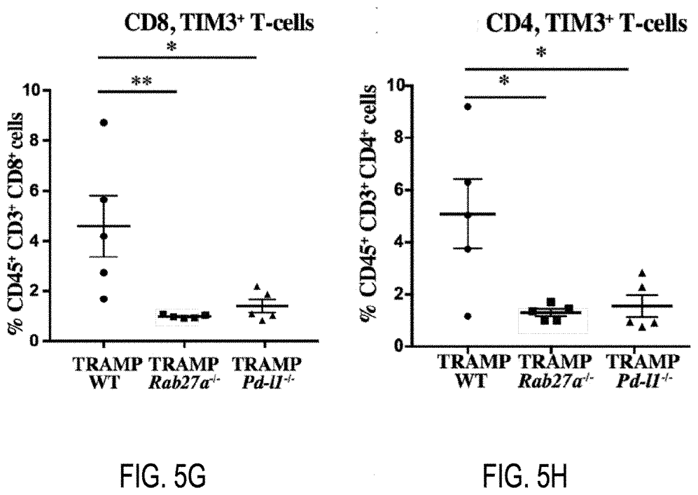

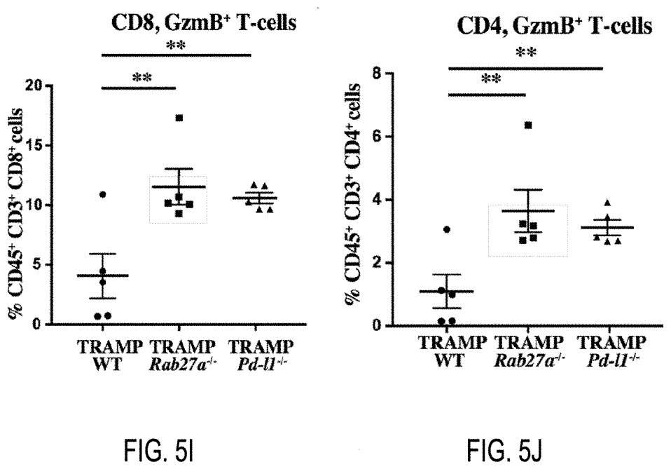

Exosomal PD-L1 suppresses priming of T cells in the draining lymph node. Next it was explored whether loss of exosomes and PD-L1 have a similar effect on the immune response to the tumor. To address this question, the immune response in the lymphoid tissues was measured following the injection of wt, Rab27a null, and Pd-l1 null TRAMP cells. At 14 days, the spleens of mice injected with either mutant cell line were significantly larger than those injected with WT cells (FIG. 5A). Immunophenotyping of the spleen showed equal percentages of CD8, CD4, and regulatory T cells across the three genotypes (FIG. 5B-5D). These data are consistent with an enhanced generalized systemic immune response in the absence of exosomes or PD-L1. In contrast, immunophenotyping of the draining lymph nodes showed striking differences between the WT and mutant animals (FIG. 5E-5J). CD8 positive cells made up a much greater fraction of the T cells following injection of the two mutant tumor cell lines relative to the WT line. The fraction of CD4 was relatively down in the mutants, while FoxP3+ T regulatory cells made up a constant fraction of the T cells. Given the relative increase in CD8 cells in the absence of exosomes or PD-L1, markers of T cell exhaustion were evaluated and activation within the CD8 and CD4 T cell populations. The fraction of CD8 and CD4 cells that were PD-1 high were trending down in mice receiving the mutant cells. Much more significant was a decrease in the percent of cells expressing the exhaustion marker Tim3 and increase in percent cells expressing the activation marker Granzyme B. Furthermore, the fraction of cells positive for the proliferation marker Ki67 was significantly up among the CD8 T cells and trending up among the CD4 T cells. These data are consistent with tumor derived exosomal PD-L1 traveling to the draining lymph node and suppressing T cell activation.

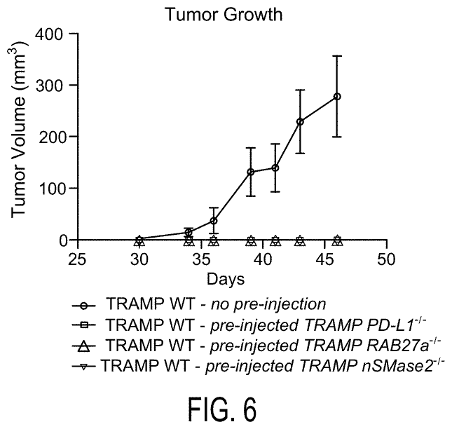

It was hypothesized that if exosomes are acting through PD-L1 to suppress the priming of T cells, then in the absence of exosomal PD-L1, immune-competent mice will not only suppress immediate growth of the mutant tumor cells, but will also develop memory toward the tumor cells. To test this hypothesis, mice injected with either Rab27a or Pd-l1 null TRAMP cells on one flank were 90 days later re-challenged with WT TRAMP cells on the other flank. Age matched mice that had not been previously injected with any tumor cells were used as control. Remarkably, WT TRAMP tumors failed to grow in any of the mice previously injected with either mutant cell line, but showed normal growth in the age matched controls (FIG. 6). Thus exposure to tumor cells lacking exosomes or PD-L1 results in a robust memory response even toward cells that secrete exosomal PD-L1. This was interpreted this to mean that once the T cells have been primed in the absence of exosomal PD-L1, they are then resistant to the suppressive effects of exosomal PD-L1.

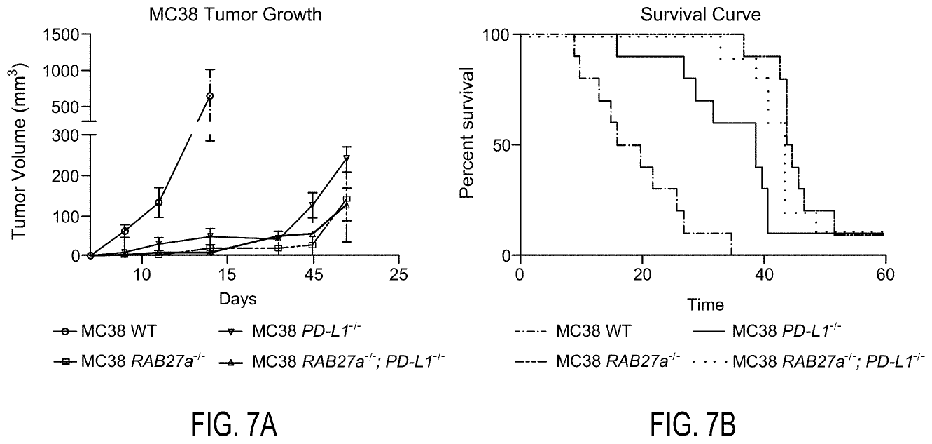

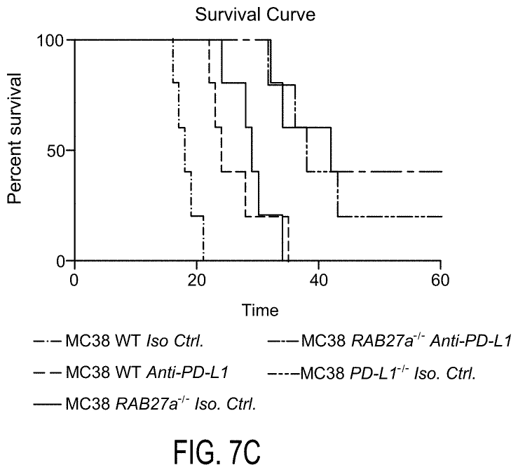

Exosomal PD-L1 promotes growth across different tumor types. Next, it was asked if the effect of exosomal PD-L1 on tumor progression is unique to the TRAMP model. To address this question, a colorectal cancer model, MC38 was evaluated. Unlike the TRAMP model, this model shows a partial response to anti-PD-L1 and anti-PD-1 therapy. Western analysis showed that MC38 cells, like TRAMP cells, secrete PD-L1. They also express low levels on the cell surface. Using identical guide RNAs as in the TRAMP model, Pd-l1 and Rab27a were knocked out in MC38 cells via CRISPR/Cas9-mediated mutagenesis. The Rab27a knockout cells showed a loss of PD-L1 in the secreted fraction confirming the packaging of PD-L1 in exosomes. Also similar to TRAMP cells, the Rab27a and Pd-l1 knockout MC38 cells showed no change in proliferation. These cells were injected in syngeneic WT mice. WT MC38 tumors grew rapidly and mice had to be euthanized starting at 9 days. Loss of either Rab27a or Pd-l1 slowed tumor growth and extended lifespan. However, unlike the TRAMP model, Pd-l1 loss appeared to have a greater effect than Rab27a loss (FIGS. 7A, 7B, and 7C).

The difference in the effect of Pd-l1 versus Rab27a loss allowed for an epistasis to address the question of whether exosomes are acting specifically through PD-L1 to influence tumor growth. If exosomes have a PD-L1 independent role, their removal should further reduce tumor growth and extend lifespan in the Pd-l1 null background. Therefore, we Rab27a; Pd-l1 double knockout MC38 cells were made. These cells grew at almost identical rate as the Pd-l1 single mutant cells. Thus, it can be concluded that exosomes are functioning predominantly, if not entirely, through their presentation of PD-L1.

The difference in the effect of Pd-l1 versus Rab27a loss on tumor growth also implied that there is an exosome independent pool of PD-L1 that is also functioning to suppress the immune response in this model. It was hypothesized that unlike exosomal PD-L1, this pool may be sensitive to anti-PD-L1. This pool could explain why unlike the TRAMP model, the MC38 model is partially responsive to anti-PD-L1 antibodies. To test this hypothesis, the effect on survival of anti-PD-L1 alone and in combination with exosome depletion was explored. Similar to the loss of Rab27a, the treatment of the mice with anti-PD-LI antibody extended survival, but not to the same degree as loss of Pd-l1 (FIG. 7C). Remarkably, the combination of Rab27a deletion and treatment with anti-PD-L1 antibodies lead to almost identical survival curve as the Pd-l1 deletion. This finding strongly supports the conclusion that while exosomal PD-L1 is resistant to anti-PD-L1, there is another pool in this model, likely cell surface PD-L1, which is both a significant suppressor of the anti-tumor immune response and sensitive to the antibodies.

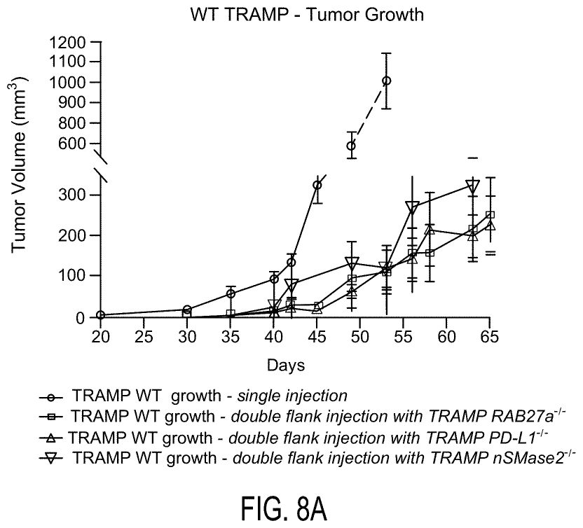

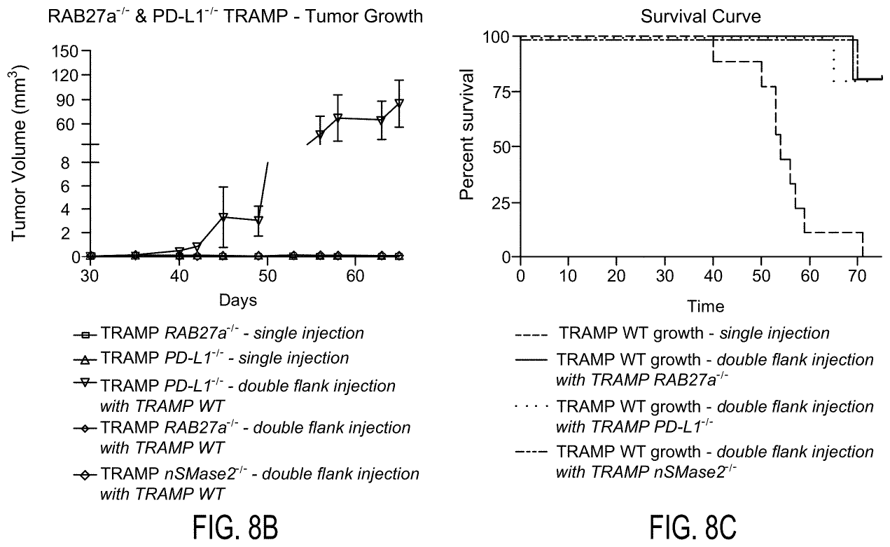

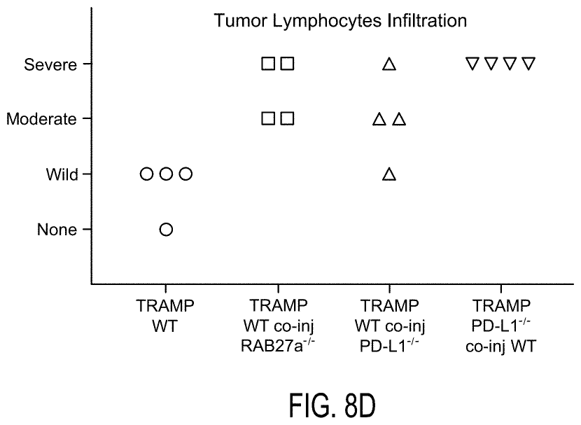

Exosomal PD-L1 deficient tumor cells can suppress WT tumor growth at a distant site. Exosomes can enter the blood and lymphatic systems and travel throughout the body potentially influencing tumor growth at distant sites. Similarly, immune cells educated at one tumor site can travel throughout the body potentially influencing growth of tumors at distant sites. Therefore, it was explored whether the simultaneous injection of WT and mutant cells at different sites would affect growth of either tumor. In particular, it was explored if exosomal PD-L1 released from WT tumor cells could promote growth of mutant cells at a distant site and/or if the T cells activated by the mutant cells suppress growth of WT cells at a distant site. To address this question, WT TRAMP cells were injected in one flank of each mouse simultaneously with Pd-l1, Rab27a, or nSMase2 null TRAMP cells on the other flank. Tumor growth on each flank was then followed over time. Remarkably, growth of the WT tumor in these double injections was dramatically reduced relative to mice where WT cells alone were injected (FIG. 8A). The effects of Pd-l1, Rab27a, or nSMase2 null cells on the distant WT tumor were similar. In contrast, the WT cells did not have any effect on the growth of Rab27a or nSMase2 null cells (FIG. 8B). However, the WT cells did promote the growth of Pd-l1 null cells. This effect was small as tumor growth was much slower than the WT counterparts. Overall, the double injected mice showed greatly extended survival relative to those that received WT TRAMP cells alone (FIG. 8C). These findings strongly supported the notion that immune cells primed in the draining lymph node of the mutant side were able to travel and attack the WT tumor cells on the opposite flank. Indeed, histological analysis of the WT tumors showed, a dramatic increase in the number of infiltrating lymphocytes when co-injected with the mutant cells on the opposite flank (FIG. 8D). Together, these data show there is communication between the tumors, with the effect of the mutant tumor being dominant over that of the WT tumor.

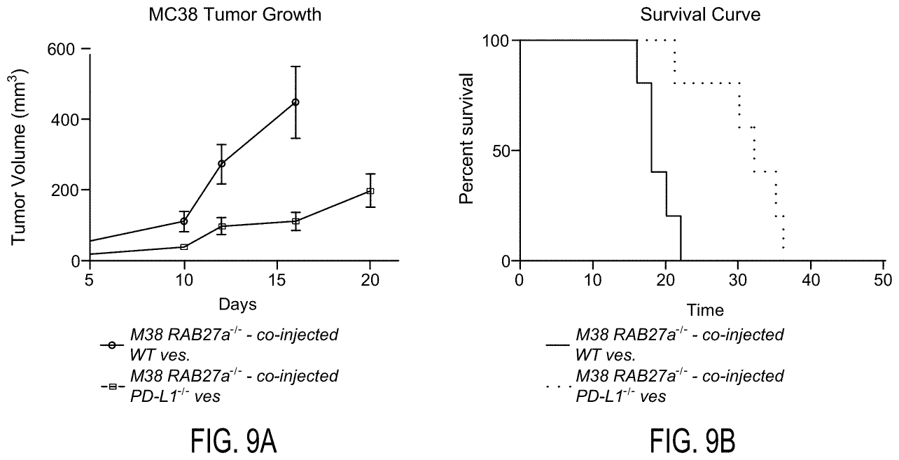

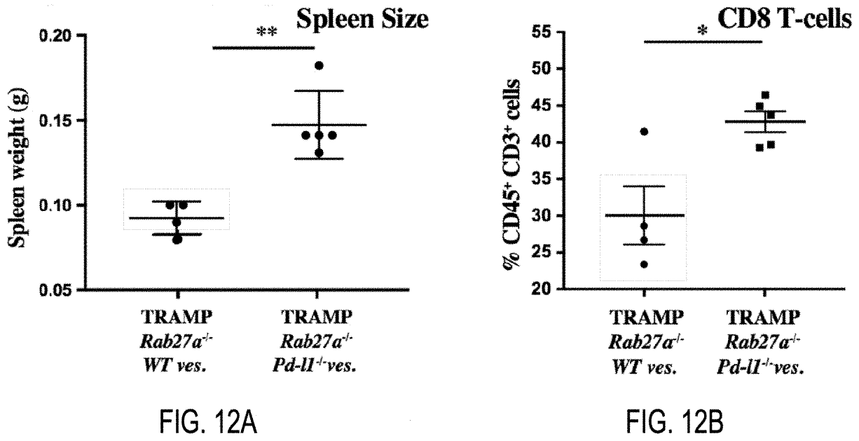

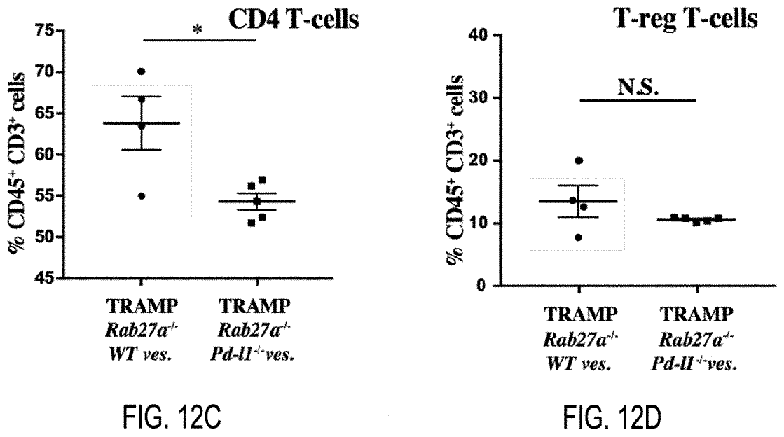

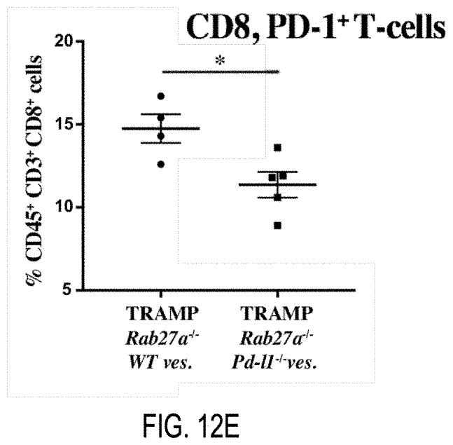

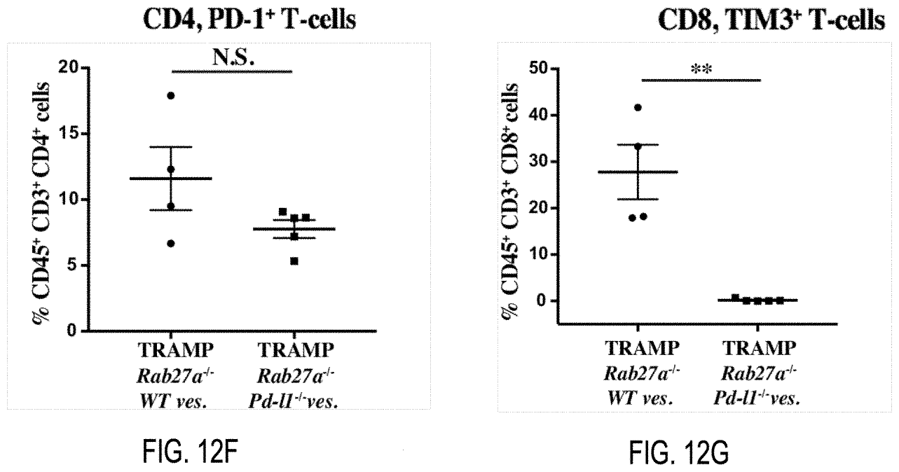

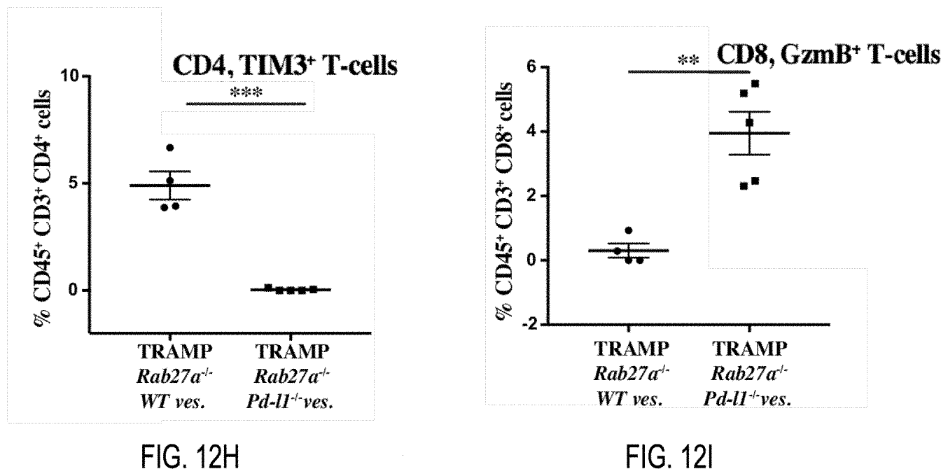

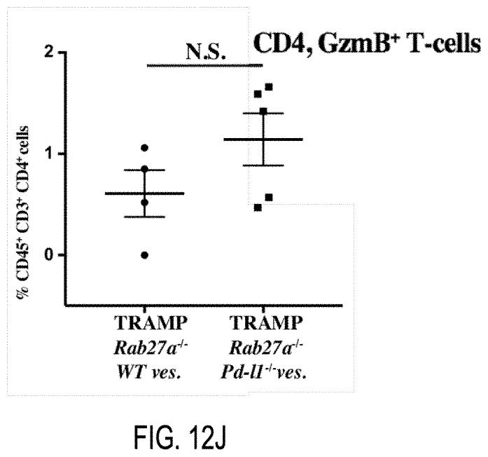

Exogenously introduced exosomal PD-L1 can rescue immune suppression and tumor growth. It was explored whether high doses of exogenously introduced exosomal PD-L1 could suppress the anti-tumor immune response and promote tumor growth. To address the effect on the immune response, Rab27a null TRAMP cells were transplanted in the flank of syngeneic mice followed by tail vein injections of in vitro collected exosomes from their WT or Pd-l1 null counterparts. Exosomes from the WT, but not Pd-l1 null cells were able to induce a systemic immunosuppression as evidenced by nearly a fifty percent reduction in spleen size (FIG. 12A). Furthermore, the WT exosomes were able to suppress the immune response in the draining lymph node of Rab27a deficient cells (FIG. 12B-12J). Compared to the Pd-l1 deficient exosomes, the WT exosomes led to a reduced CD8/CD4 ratio, while having little effect on the T-reg cells. More importantly, they led to an increase in the fraction of cells expressing high levels of the exhaustion markers PD-1 and TIM3 and low levels of the activation marker Gramzyme B. These findings paralleled the findings seen in mice transplanted with WT versus Rab27a null cells. To address the effect on the tumor growth, the MC38 model was employed, given its more rapid growth characteristics. Consistent with the immune suppression seen in the TRAMP model, injection of exosomes collected from WT, but not Pd-l1 deficient MC38 cells, promoted tumor growth and reduced survival (FIGS. 9A and 9B). These results confirm that exosomes are functioning through PD-L1 to suppress the anti-tumor immune response and thus promote tumor growth.

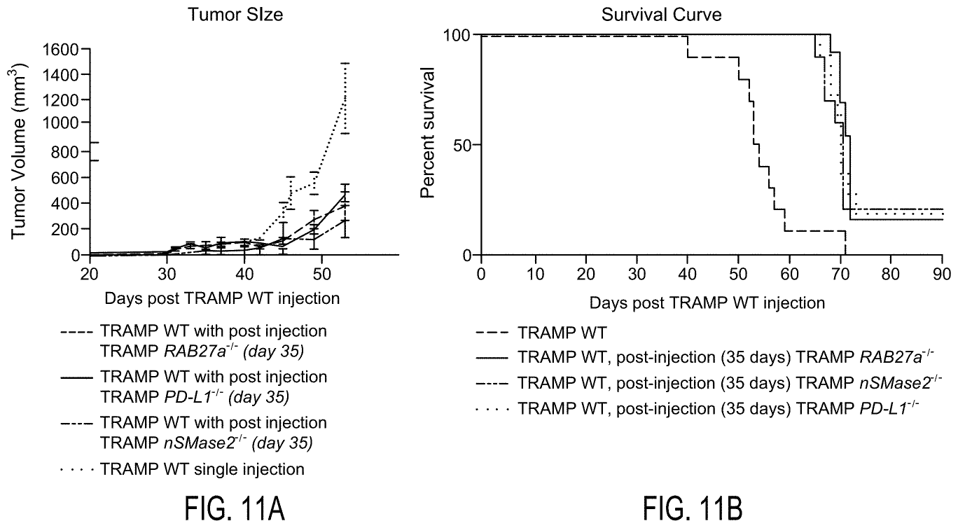

Cancer immunization. The ability of cancer cells lacking suppressive EVs to promote immune responses against resident tumors was demonstrated. 10⁶ wild type TRAMP cells were injected in one flank of mice and allowed to to grow for 35 days, resulting in the formation of a tumor mass. At 35 days the same mice received, in the opposite flank of the resident tumor, an injection, of Rab27a null, sNmase2 null, PD-L1 null mutant TRAMP cells or control vehicle. In mice that did not receive the mutant TRAMP cells, the resident tumor continued to grow aggressively. In contrast, those mice that received the Rab27a-, sNmase2-, or PD-L1-null TRAMP cells showed substantially slower tumor growth (FIG. 11A) and improved survival (FIG. 11B). These results show that engineered cancer cells with impaired production of suppressive molecules (PD-L1 null) or impaired production of EVs (Rab27a null, sNmase2 null) can stimulate effective immune responses against resident tumors.

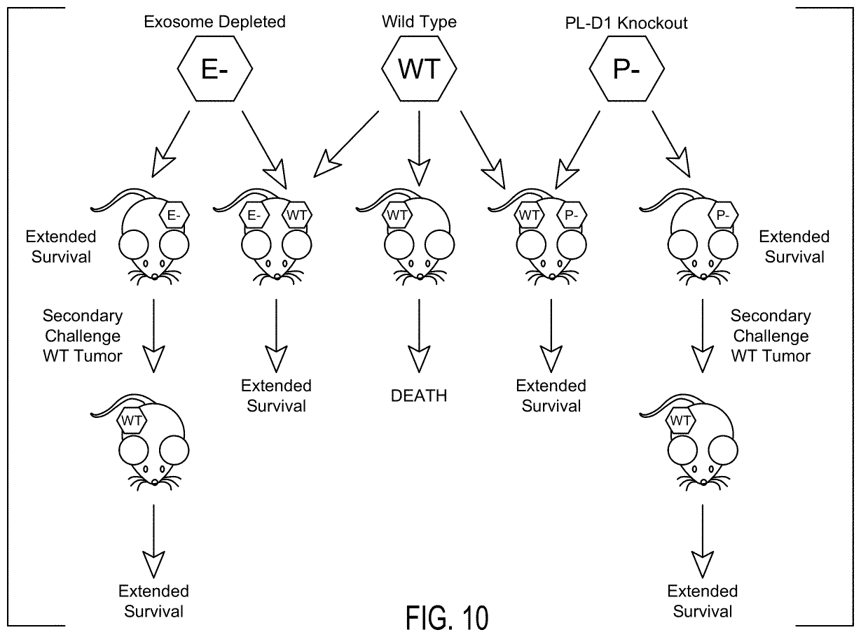

Discussion. All together, these data uncover a key role for exosomal PD-L1 in enabling cancer cells to evade anti-tumor immunity. Indeed, in the presence of exosomal PD-L1, T cells in the tumor's draining lymph node express markers of exhaustion and the spleens are smaller. Genetically blocking exosome biogenesis or deleting Pd-l1 reverses the phenotype by strongly promoting T-cells activation, proliferation and killing potential. This effect is reversed again with the introduction of exogenous exosomal PD-L1. Therefore, tumor exosomes have the ability to travel to the draining lymph node, where they present PD-L1 inhibiting T cell activation. Remarkably, blocking the release of exosomal PD-L1 not only suppressed growth of the local tumor cells, but also blocked wild-tumor cells injected at a distant site either simultaneously or many months later. Therefore, enabling T cell activation at the local lymph node leads to a durable systemic immune response that is no longer affected by the secretion of exosomal PD-L1. The end result is extended survival of the afflicted mice (FIG. 10).

A role for EVs, including exosomes, in tumor progression has been proposed in a number of settings. EVs can carry tumor antigens. When taken up by dendritic cells these antigens can be presented inducing an immune response. As such, EVs were originally thought to have an anti-tumor effect. However, more recent studies have suggested a number of immunosuppressive effects. For example, EVs can inhibit dendritic cell maturation, NK cell function, and directly kill CD8 T cells. EVs have also been shown to promote directional migration of tumor cells, home tumor cells to lymph nodes, induce neovascularization and leakiness of tumor vessels, and even establish pre-metastatic niches. The proposed mechanisms behind these various functions have been largely speculative.

Herein is presented extensive evidence that EVs, specifically exosomes, can function to promote tumor progression by presenting PD-L1. In vitro, exosomes suppressed T cells in a PD-L1 dependent fashion. In vivo, the removal of tumor exosomes from TRAMP cells using two independent genetic mutations recapitulated the effects of deleting Pd-l1. These recapitulated effects included suppression of tumor growth, increased cellularity of the spleen, and the activation of a T cell response in lymph nodes with almost identical effects on the various activation, exhaustion, and proliferation markers. All these outcomes were reversed with the injection of in vitro collected exosomes carrying PD-L1. In the absence of PD-L1, the exogenously introduced exosomes had little effect. This demonstrates that presentation of PD-L1 is the major mechanism by which exosomes promote cancer progression.

This role for exosomal PD-L1 is not limited to the TRAMP model. Removal of exosomes in the colorectal MC38 model also suppressed tumor growth and extended survival. Once again, the effect was dependent on PD-L1 as the removal of exosomes had no additional effect in the Pd-l1 null background. Interestingly though, unlike the TRAMP model, the loss of exosomes alone did not have as much of an impact as Pd-l1 loss, suggesting a combined role of exosomal and cellular PD-L1 in the MC38 model. Remarkably, combining exosome loss with anti-PD-L1 treatment extended survival of these mice to a similar degree as removing PD-L1 altogether. These data show that in the MC38 model, both exosomal and cellular PD-L1 play an important role in promoting tumor progression with the later, but not the former, being sensitive to anti-PD-L1 therapy.

The data demonstrate that targeting both cell surface and exosome presentation of PD-L1 should be considered in any therapeutic strategy. The TRAMP model is resistant to current anti-PD-L1 and anti-PD-1 antibody blockade. However, the deletion of Pd-l1 in the tumor cells had a striking effect. Similarly, although the MC38 model shows partial responsiveness to anti-PD-L1/PD-1 therapy, deletion of the Pd-l1 gene has a greater effect. These data are all consistent with exosomal PD-L1 being resistant to current anti-PD-L1/anti-PD-1 therapy.

As shown herein, inhibition of exosome secretion at one tumor site can lead to a systemic and durable immune response against distant tumor sites or secondary tumor challenges. This observation is reminiscent of the abscopal effect, originally seen in patients treated with irradiation. In particular, irradiation of the primary tumor can lead to secondary regression of metastases. It is thought that this phenomenon is driven by activation of anti-tumor immune response and preclinical studies combining irradiation with immunosuppression are underway. Results herein suggest that localized anti-exosomal therapy combined with systemic anti-PD-L1/PD-1 blockade could synergize to induce a systemic immune response against multiple tumor sites simultaneously.

Genetic approaches were used herein to dissect the role of exosomal PD-L1 in tumor progression by deleting two important exosomal biogenesis genes: Rab27a and nSMNase2. The deletion of Rab27a led to loss of all exosomes as measured by markers (CD63, HRS), particle tracking, and electron microscopy. The deletion of nSMNase2 led to a loss of a majority, but not all exosomes, as measured by the same assays. Therefore, both enzymes represent therapeutic targets. In summary, the results presented herein show that exosomal PD-L1 is a major regulator of tumor progression through its ability to suppress T cell activation in draining lymph nodes and that its inhibition can lead to a long-lasting, systemic anti-tumor immunity.

Methods

CRISPR-Cas9-mediated gene disruption. sgRNA oligonucleotides SEQ ID NO: 3-14, were cloned into pSpCas9(BB)-2A-GFP according to the known protocol. For each gene disrupted, two different guides were simultaneously transfected. 1 ug of each vector was transfected using FUGENE HD(™) (Promega). Pd-l1 null, Rab27a null and nSMase2 null clones were obtained by GFP+ single cell cloning, 48 hours post transfection. Knockout clones were identified either by western (Rab27a) of by flow cytometry analysis for cell surface PD-L1.

Nanoparticle Tracking Analysis. Equal amount of cells was seeded in KSR media 24 hours before collection. Media was pre-processed at 300 g for 10 minutes at room temperature, followed by 2 k g for 20 min at 4° C. then 12 k g for 40 minutes at 4° C. The processed media was analyzed on a NANOSIGHT LM10(™) (Nanosight limited).

Tumor cells injections Mice were injected with a million TRAMP-c2 wt or TRAMP-c2 Pd-l1 null or TRAMP-c2 Rab27a null or TRAMP-c2 nSMase2 null cells. Mice were injected with a million MC38 wt or MC38 Pd-l1 null or MC38 Rab27a null cells. Mice were considered “end stage” when the tumor was reaching 2 cm in at least one dimension. Tumor growth was monitored three times a week by measuring tumor length and width. Tumor volume was calculated according to the following equation: length×width×0.5×width.

Where indicated mice were treated with exosomes injected IV in the tail vein. 15 million cells were seeded in five 15 cm dishes (Corning CLS430599), and cultured for 48 hours. Vesicles were isolated as a 100 k g pellet as described above. MC38 vesicles were resuspended in 1 ml PBS and TRAMP vesicles in 600 μl. Each mouse was injected with 100 μl of PBS containing vesicles.

1.-51. (canceled)

52. A method of treating cancer in a subject having a tumor or other neoplastic condition, comprising:
administering to the subject a therapeutically effective amount of an engineered cell;
wherein the engineered cell is derived from or is of the same immunologic phenotype as the cells of the tumor or other neoplastic condition;
wherein the cell is are engineered to have reduced suppressive EV activity or reduced EV production, relative to like, non-engineered cells; and
wherein the host immune system response to the administered engineered cells provides an immune response against resident, non-engineered cells of the tumor or other neoplastic condition.

53. The method of claim 52, wherein
the engineered cell is a cancer cell of a cancer selected from the group consisting of bladder cancer, brain cancer, breast cancer, cervical cancer, colorectal cancer, esophageal cancer, head and neck cancer, kidney cancer, lung cancer, leukemia, lymphoma, myeloma, ovarian cancer, pancreatic cancer, prostate cancer, sarcoma, and skin cancer.

54. The method of claim 52, wherein
the engineered cell is derived from autologous cells of the tumor or other neoplastic condition that have been obtained from the subject.

55. The method of claim 52, wherein
the engineered cell is an allogenic cell.

56. The method of claim 52, wherein
the engineered cell is engineered to have impaired expression of one or more suppressive molecules, compared to like non-engineered cells.

57. The method of claim 56, wherein
the one or more suppressive molecules is selected from the group consisting of PD-L1, PD-1, PD-2, adenosine A2A receptor, B7-H3, B7-H4, BTLA, CTLA-4, Indoleamine 2,3-dioxygenase, Killer-cell Immunoglobulin-like Receptor, Lymphocyte Activation Gene-3, NOX-2, PD-1, TIM-3, and V-domain Ig suppressor of T cell activation.

58. The method of claim 52, wherein
the engineered cell is engineered to have a reduced capacity for EV production, packaging of suppressive molecules, biogenesis or secretion, relative to like, non-engineered cells.

59. The method of claim 58, wherein,
the engineered cell comprises a cell wherein one or more genes for EV production, packaging of suppressive molecules, biogenesis, or secretion genes is inhibited.

60. The method of claim 58, wherein
the one or more genes is selected from the group consisting of Rab27a, Rab27b, nSMase2, a gene coding for proteins of the ESCRT0, ESCRT1, ESCRT2, or ESCRT3 complexes, nSMase2, a gene coding for proteins of the Soluble N-ethylmaleimide-sensitive factor attachment protein receptor (SNARE) complexes, ALIX, TSG101, HRS, syntenin, ubiquitin, clathrin, VPS32, VPS, SNAP23, VAMP3, VAMP7, YKT6, RAB-11, RAB-35, RAB-5, and RAB-7.

61. The method of claim 52, wherein
the engineered cells are further engineered to express one or more factors which enhance immunogenic responses.

62. The method of claim 52, wherein
the one or more factors which enhance immunogenic responses is selected from the group consisting of granulocyte-macrophage colony-stimulating factor, cyclic di-guanylate, alphafetoprotien, carcinoembryogenic protein, CA-125, MUC1, epithelial tumor antigen, tyrosinase, melanoma associated antigen, IL-18, IL-6, hyper IL-6, IL-11, hyper IL-11, IL15, and IL15α.

63. A method of treating an immune-related condition in a subject, comprising administration to the subject of a therapeutically effective amount of exogenously-produced EV's bearing one or more immunosuppressive molecules.

64. The method of claim 63, wherein
the immune-related condition comprises an inflammatory condition, an autoimmune condition, or the need for immunosuppression associated with the receipt of a transplant.

65. The method of claim 63, wherein
the immune-related condition comprises an aautoimmune condition selected from the group consisting of arthritis, multiple sclerosis, inflammatory bowel disease, Crohn disease, lupus, autoimmune uveitis, type I diabetes, bronchial asthma, lupus, retinitis, pancreatitis, cardiomyopathy, pericarditis, colitis, glomerulonephritis, lung inflammation, esophagitis, gastritis, duodenitis, ileitis, meningitis, encephalitis, encephalomyelitis, transverse myelitis, cystitis, urethritis, mucositis, lymphadenitis, dermatitis, hepatitis, osteomyelitis, psoriasis, scleroderma, dermatomyositis, epidermolysis bullosa, and bullous pemphigoid.

66. The method of claim 65, wherein
the immune-related condition comprises the need for immunosuppression associated with the receipt of a transplant, wherein the transplant comprises a graft selected from the group consisting of an organ, tissue, cells, kidney, heart, lung, liver, skin, cornea, intestine, pancreas, limb, digit, bone, ligament, cartilage, and tendon.

67. The method of claim 63, wherein
the one or more suppressive molecules is selected from the group consisting of PD-L1, PD-1, PD-2, adenosine A2A receptor, B7-H3, B7-H4, BTLA, CTLA-4, Indoleamine 2,3-dioxygenase, Killer-cell Immunoglobulin-like Receptor, Lymphocyte Activation Gene-3, NOX-2, PD-1, TIM-3, and V-domain Ig suppressor of T cell activation.

68. The method of claim 63, wherein
the one or more suppressive molecules is an immunosuppressive peptide, selected from the group consisting of Antamides, Collutellin A, Cyclosporine A, Didemnin A/B, FK506 (tracrolimus), Ascomycin (pimecrolimus, SDZ ASM 981), Homophymines, Geodiamolides H, Hymenistatin, Charybdotoxin I, Curcacycline B, Cyclolinopeptide A/B Iberiotoxin, Kalata Bl, Magatoxin, and Kaliotoxin.

69. The method of claim 63, wherein
the exogenously-produced EV's comprise exosomes.

70. The method of claim 63, wherein
the exogenously-produced EV's are produced by cultured cells.

71. A method of treating cancer in a subject, comprising
administering to the subject a therapeutically effective amount of a suppressive EV inhibitor,
wherein the suppressive EV inhibitor inhibits the suppressive activity of suppressive EVs, inhibits the packaging of suppressive molecules in EVs, and/or inhibits the production, biogenesis, or secretion of suppressive EVs.

72. The method of claim 71, wherein
the suppressive EV inhibitor inhibits the activity of one or more suppressive molecules present on or within suppressive EVs, wherein the one or more suppressive molecules is selected from the group consisting of PD-L1, PD-1, PD-2, adenosine A2A receptor, B7-H3 (CD276), B7-H4 (VTCN1), BTLA, CTLA-4, Indoleamine 2,3-dioxygenase, Killer-cell Immunoglobulin-like Receptor, Lymphocyte Activation Gene-3,NOX-2, PD-1, TIM-3, and V-domain Ig suppressor of T cell activation.

73. The method of claim 71, wherein
the suppressive EV inhibitor disrupts the expression of suppressive molecules present on or within suppressive EVs, wherein the one or more suppressive molecules is selected from the group consisting of PD-L1, PD-1, PD-2, adenosine A2A receptor, B7-H3 (CD276), B7-H4 (VTCN1), BTLA, CTLA-4, Indoleamine 2,3-dioxygenase, Killer-cell Immunoglobulin-like Receptor, Lymphocyte Activation Gene-3,NOX-2, PD-1, TIM-3, and V-domain Ig suppressor of T cell activation.

74. The method of claim 71, wherein
the suppressive EV inhibitor inhibits the packaging of suppressive molecules in EVs.

75. The method of claim 71, wherein
the suppressive EV inhibitor inhibits the production, biogenesis, or secretion of EVs.

76. The method of claim 75, wherein
the suppressive EV inhibitor inhibits the expression of an EV production, biogenesis, or secretion gene.

77. The method of claim 76, wherein
the EV production gene is selected from the group consisting of Rab27a, Rab27b, a gene coding for an endosomal sorting complex required for transport (ESCRT) element, nSMase2, is any of the genes coding for proteins of the Soluble N-ethylmaleimide-sensitive factor attachment protein receptor (SNARE) complexes, ALIX, TSG101, HRS, syntenin, ubiquitin, clathrin, VPS32, VPS, SNAP23, VAMP3, VAMP7, YKT6, RAB-11, RAB-35, RAB-5, and RAB-7.

78. The method of claim 71, wherein
the suppressive EV inhibitor is a small molecule.

79. The method of claim 78, wherein
the small molecule is selected from the group consisting of an inhibitor of Rab27a, Nexinhib20, an inhibitor of nSMase2, cambinol, GW4869, 2,6-Dimethoxy-4-(5-Phenyl-4-Thiophen-2-yl-1H-Imidazol-2-yl)-Phenol (DPTIP), tipifarnib, neticonazole, climbazole, isoproterenol, ketoconazole, mitotane, triademenol, pentetrazol, Cannabidiol, simvastatin, Brefeldin A, tunicamycin, dimethyl amiloride, Monensin, chloramidine, and bisindolylmaleimide-I.

80. The method of claim 71, wherein
the suppressive EV inhibitor of the invention is co-administered with one or more immunotherapy agents.

81. The method of claim 80, wherein
the one or more immunotherapy agents comprises an immune checkpoint inhibitor, a cellular immunotherapy agent, an agent which primes immune cells in vivo, a cytokine, or an antibody directed to a cancer-associated antigen.
